(19) United States
(12) Patent Application Publication
(10) Pub. No.: US 2021/0156865 A1
(43) Pub. Date: May 27, 2021

(54) METHODS FOR ASSESSING EFFICACY OF MALT1 INHIBITORS USING AN NF-KB TRANSLOCATION ASSAY

(71) Applicant: Janssen Pharmaceutica NV, Beerse (BE)

(72) Inventors: Alexander Babich, Southampton, PA (US); Sriram Balasubramanian, San Diego, CA (US); Jing Cao, Maple Glen, PA (US); Gourav Choudhary, Norristown, PA (US); Bradley W. Foulk, Chalfont, PA (US); Liat Izhak, Ambler, PA (US); Ulrike Philippar, Antwerpen (BE); Nele Vloemans, Oostmalle (BE)

(21) Appl. No.: 16/953,580

(22) Filed: Nov. 20, 2020

Related U.S. Application Data
(60) Provisional application No. 62/939,022, filed on Nov. 22, 2019.

(51) Int. Cl. G01N 33/574; A61P 35/02; A61K 31/4709; A61K 45/06

(52) U.S. Cl. CPC G01N 33/57492; A61K 45/06; A61K 31/4709; A61P 35/02

(57) ABSTRACT

Methods and reagents for determining treatment efficacy of a MALT1 inhibitor in a human subject are described. The method involves determining NF-κB nuclear translocation in stimulated PBMCs of a blood sample obtained from the subject. The method provides information for guiding treatment decisions for those subjects receiving a MALT1 inhibitor therapy, improves the accuracy of optimizing therapy, reduces toxicity, and/or monitors the efficacy of therapeutic treatment.

### CROSS REFERENCE TO RELATED APPLICATION

This application claims priority to U.S. Provisional Application No. 62/939,022 filed on Nov. 22, 2019, the disclosure of which is incorporated herein by reference in its entirety.

### FIELD OF THE INVENTION

The present application relates to an NF-κB translocation assay and the use of such assay in predicting the efficacy of MALT1 (mucosa-associated lymphoid tissue lymphoma translocation 1) inhibitor and designing a method of treatment in a subject. In particular, the application relates to an assay for assessing the pharmacodynamic effects of a MALT1 inhibitor in a subject by measuring a suppression of NF-1f nuclear translocation in stimulated peripheral blood mononuclear cells (PBMCs) of the subject.

### BACKGROUND OF THE INVENTION

The nuclear factor-kappaB transcription factor (NF-κB) complex regulates genes important in cell proliferation, survival and drug resistance. The NF-κB transcription factor family in mammals consists of five proteins, p50, p52, p65, Rel-B and c-Rel, which associate with each other to form distinct transcriptionally active homo- and heterodimeric complexes. In unstimulated cells, the NF-κB complex is held in an inactivated state in the plasma by the inhibitor of KB (IκB). When activated by signals, usually coming from the outside of the cell, the IκB kinase (IKK) phosphorylates the IκB, which leads to the degradation of IκB and the release of NF-κB complex for translocation to the nucleus and activation of target genes. Nuclear translocation of the NF-κB complex is a critical step in the coupling of extracellular stimuli to the transcriptional activation of specific target genes.

Aberrant activity of the NF-κB pathway is known to be integral to the pathogenesis of many diseases, such as different types of B-cell non-Hodgkin's lymphoma (NHL) and chronic lymphocytic leukemia (CLL). Constitutive activation of NF-1B signaling is the hallmark of diffuse large B cell lymphoma of the activated B cell-like subtype (ABC-DLBCL), which is the more aggressive form of diffuse large B cell lymphoma (DLBCL). DLBCL is the most common form of non-Hodgkin's lymphoma (NHL), accounting for approximately 25% of lymphoma cases while ABC-DLBCL comprises approximately 40% of DLBCL. NF-κB pathway activation can be driven by mutations of signaling components, such as mutations in one or more genes of CD79A, CD79B, CARD11, MYD88 and A20, in ABC-DLBCL patients.

MALT1 (mucosa-associated lymphoid tissue lymphoma translocation 1) is a key mediator of the classical NF-κB signaling pathway. MALT1 affects NF-κB signaling by two mechanisms: (1) MALT1 functions as a scaffolding protein and recruits NF-κB signaling proteins such as TRAF6, TAB-TAK1 or NEMO-IKKα/β; and (2) MALT1, as a cysteine protease, cleaves and thereby deactivates negative regulators of NFKB signaling, such as RelB, A20 or CYLD. The ultimate endpoint of MALT1 activity is the nuclear translocation of the NF-κB transcription factor complex and activation of NF-κB signaling.

The API2-MALT1 oncoprotein is a potent activator of the NF-κB pathway. It comprises the amino terminus of inhibitor of apoptosis 2 (API2 or cIAP2) fused to the carboxy terminus of MALT1 and is created by chromosomal translocation in MALT lymphoma. API2-MALT1 mimics ligand-bound TNF receptor and promotes TRAF2-dependent ubiquitination of RIP1, which acts as a scaffold for activating canonical NF-κB signaling. Furthermore, API2-MALT1 has been shown to cleave and generate a stable, constitutively active fragment of NF-κB-inducing kinase (NIK) thereby activating the non-canonical NF-κB pathway.

It is believed that MALT1 inhibition may: 1) allow for suppression of NF-κB activity in participants with tumors resistant to alternative pathway inhibiting medications, 2) augment suppression when combined with other NF-κB inhibitors, and 3) be tumoricidal in malignancies with certain genetic mutations. The use of BTK inhibitors, for example Ibrutinib, provides clinical proof-of-concept that inhibiting NF-κB signaling in ABC-DLBCL is efficacious. MALT1 is downstream of BTK in the NF-κB signaling pathway, and a MALT1 inhibitor could target ABC-DLBCL patients not responding to Ibrutinib, such as patients with CARD11 mutations, as well as treat patients that acquired resistance to Ibrutinib. Small molecule inhibitors of MALT1 have demonstrated efficacy in preclinical models of ABC-DLBCL.

In addition to lymphomas, MALT1 has also been shown to play a critical role in innate and adaptive immunity. Studies have suggested that inhibiting MALT1 may help treat autoimmune disease. For example, it was reported that pharmacological inhibition of MALT1 protease activity protects mice in a mouse model of multiple sclerosis.

A MALT1 inhibitor (MI-2) was shown to suppress nuclear translocation of NF-κB proteins in CLL cells. The assay was conducted by measuring the nuclear levels of NF-κB proteins (p50 and RelB) in CLL cells treated with the MALT1 inhibitor in vitro, via an enzyme-linked immunosorbent assay (ELISA). The MALT1 inhibitor (MI-2) was also shown to significantly reduce the expression of six known NF-κB target genes (CCND2, BCL2A, CCL3, CCL4, RGS1, and TNF) in the CLL cells treated with the MALT1 inhibitor in vitro, as measured by quantitative RT-PCR. Treatment with a MALT1 inhibitor showed a significant reduction in an NF-κB target gene signature in two ABC DLBCL lines tested. However, in clinical context, given the low number of tumor cells when compared to normal cells and the heterogeneity of cancer cells, the detection of nuclear translocation of NF-κB or the measurement of NF-kB target gene expression in the tumor cells presents a major challenge.

There is a need for a reproducible and relatively inexpensive method to assess the pharmacodynamic effects of a MALT1 inhibitor in a clinical context, and to determine whether a subject is responsive to a MALT1 inhibitor treatment.

### BRIEF SUMMARY OF THE INVENTION

The present application relates to a method of assessing the pharmacodynamic effects of a MALT1 inhibitor by measuring the degree of NF-κB nuclear translocation in a subject's sample. The nuclear translocation may be measured by determining the level of any one of the NF-kB subunits p50, p52, RelA, RelB and c-Rel in the nucleus of a subject's cell that is exposed to a MALT1 inhibitor. The methods disclosed herein can be used to determine or predict a response to a MALT1 inhibitor in a subject in need of a treatment of a MALT-mediated disease, such as lymphoma or an autoimmune disease. As such, a method of the present application provides information for identifying subjects responsive to a MALT1 inhibitor, guiding treatment decisions for those subjects receiving a MALT1 inhibitor therapy and/or monitoring the efficacy of an ongoing MALT1 inhibitor therapy.

In one general aspect of the application, a method of predicting a response to a MALT1 inhibitor in a subject comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; and (c) comparing the changed level of NF-kB nuclear translocation in the subject's test sample to the changed level in the control sample, wherein a decrease in the changed level of NF-kB nuclear translocation in the test sample is predictive of a positive response to the MALT1 inhibitor in the subject.

In another embodiment, a method of monitoring the efficacy of an ongoing MALT1 inhibitor therapy in a subject comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; and (c) comparing the changed level of NF-kB nuclear translocation in the subject's test sample to the changed level in the control sample, wherein a decrease in the changed level of NF-kB nuclear translocation in the test sample is indicative of efficacy of MALT1 inhibitor therapy in the subject.

In another embodiment, a method of treating a cancer or a MALT1-mediated disease in a subject comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; (c) comparing the changed level of NF-kB nuclear translocation in the subject's test sample to the changed level in the control sample; and (d) administering a lower dose of MALT1 inhibitor to the subject if the test sample displays a decrease in the changed level of NF-kB nuclear translocation, and administering a higher dose of MALT1 inhibitor to the subject if the test sample does not display a decrease in the changed level of NF-kB nuclear translocation.

In another embodiment, a method of treating a cancer or a MALT1-mediated disease in a subject comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; (c) comparing the changed level of NF-kB nuclear translocation in the subject's test sample to the changed level in the control sample; and (d) administering an effective amount of MALT1 inhibitor to the subject if the test sample displays a decrease in the changed level of NF-kB nuclear translocation.

In another embodiment, a method of designing a drug regimen to treat cancer or a MALT1-mediated disease in a subject comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; (c) comparing the changed level of NF-kB nuclear translocation in the subject's test sample to the changed level in the control sample; and (d) administering a second therapeutic agent to the subject if the test sample does not display a decrease in the changed level of NF-kB nuclear translocation.

In another embodiment, a method of modifying the dose and/or frequency of dosing of a MALT1 inhibitor in a subject suffering from cancer or a MALT1-mediated disease comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; (c) comparing the changed level of NF-kB nuclear translocation in the subject's test sample to the changed level of the control sample; and (d) reducing the dosing frequency of a MALT1 inhibitor if the test sample displays a decrease in the changed level of NF-kB nuclear translocation, and increasing the dosing frequency of a MALT1 inhibitor if the test sample does not display a decrease in the changed level of NF-kB nuclear translocation.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1B show graphs demonstrating percentage of T cells in normal blood (FIG. 1A) or NHL blood (FIG. 1B) expressing CD69 over time upon stimulation with anti-CD3 and anti-CD-28 antibodies in cells treated with Compound A versus a control (DMSO).

FIG. 2 is a graph demonstrating the fold change in frequency of total T cells with nuclear enrichment of p50 (a subunit of NF-κB) in an NHL blood sample treated with increasing concentrations of Compound A.

FIG. 3 shows a graph demonstrating the p50 nuclear index in unstimulated and anti-IgM stimulated B cells treated with Compound A versus control (DMSO).

FIG. 4 shows a graph demonstrating percentage of nuclear p50 in CLL B cells in unstimulated and anti-IgM stimulated cells treated with Compound A versus control (DMSO).

FIG. 5 show a graph demonstrating percentage of nuclear p50 in CLL T cells in unstimulated and anti-IgM stimulated cells treated with Compound A versus control (DMSO).

FIGS. 6A-6B show graphs demonstrating CXCL10 expression levels in NHL (FIG. 6A) and CLL (FIG. 6B) donor samples treated with Compound A.

FIG. 7 shows graphs demonstrating IL2 expression levels in purified T-cells and purified peripheral blood mononuclear cells (PBMCs) from NHL donor samples treated with Compound A.

FIGS. 8A-8D show graphs demonstrating NF-kB2 (FIG. 8A), TNFSF10 (FIG. 8B), APOE (FIG. 8C), and PYCARD (FIG. 8D) expression levels in purified PBMCs from NHL donor samples treated with Compound A, and the PBMCs were unstimulated.

FIGS. 9A-9D show graphs demonstrating NF-kB translocation in T cells from peripheral blood of donors with NHL upon ex vivo stimulation with different stimulating agents: the nuclear index in T cells for NF-kB nuclear translocation corrected for baseline levels in unstimulated samples (NF-kB Δnuclear Index) for T cells in the control blood sample treated with DMSO (Control) and test blood sample treated with Compound A (Compound A) stimulated with anti-CD3 and anti-CD28 antibodies (FIG. 9A) and phorbol myristate acetate (PMA)/ionomycin (FIG. 9B); and the mean values of NF-kB Δnuclear Index in Compound A treated sample normalized to that of the Control and represented as percentage of inhibition for samples stimulated with anti-CD3 and anti-CD28 antibodies (FIG. 9C) and phorbol myristate acetate (PMA)/ionomycin (FIG. 9D). Data in C and D are mean with standard error of means.

### DEFINITIONS

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

As used herein, the term “about” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 10%, whichever is larger.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

The term “predicting” is used herein to refer to the likelihood that a patient will respond either favorably or unfavorably to a drug (therapeutic agent) or set of drugs or a therapeutic regimen. In one embodiment, the prediction relates to whether and/or the probability that a patient will survive or improve following treatment, for example treatment with a particular therapeutic agent.

The term “sample,” as used herein, refers to a composition that is obtained or derived from a subject of interest that contains a cellular and/or other molecular entity that is to be characterized and/or identified, for example based on physical, biochemical, chemical and/or physiological characteristics.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

As used herein, a “stimulated cell,” “stimulated sample,” “stimulated test blood sample,” or “stimulated control blood sample” refers to a cell, sample, test blood sample or control blood sample, respectively, that has been exposed to or treated with one or more stimulating agents in vitro prior to being analyzed or measured by a method of the application. A stimulating agent can be any agent that activates the NF-kB pathway.

As used herein, a “test sample” or “test blood sample” refers to a sample or blood sample that has been exposed to a MALT1 inhibitor. As used herein, a “control sample” or “control blood sample” refers to a sample or blood sample that has not been exposed to a MALT1 inhibitor or is known to be no longer affected by a MALT1 inhibitor.

As used herein, “treat”, “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subject have previously had the disorder, or limiting or preventing recurrence of symptoms in subjects that were previously symptomatic for the disorder.

As used herein, an “unstimulated cell,” “unstimulated sample,” “unstimulated test blood sample,” or “unstimulated control blood sample” refers to a cell, sample, test blood sample or control blood sample, respectively, that has not been exposed to or treated with one or more stimulating agents in vitro prior to being analyzed or measured by a method of the application. A stimulating agent can be any agent that activates the NF-kB pathway.

The term “whole blood” refers to any whole blood sample obtained from an individual. Typically, whole blood contains all of the blood components, e.g., cellular components and plasma. Methods for obtaining whole blood from mammals are well known in the art.

### Methods

Disclosed herein are methods to monitor the NF-κB nuclear translocation in a subject who has been administered with a MALT1 inhibitor. The present invention also provides a MALT1 inhibitor for use in a method of treatment or diagnosis. For each and every method in this disclosure, the invention provides a further embodiment relating to a MALT1 inhibitor for use in that therapeutic or diagnostic method.

By employing such methods, a response to the MALT1 inhibitor or the pharmacodynamic effects (e.g., the relationship of drug concentration or dose and pharmacologic or toxicologic responses) of a MALT1 inhibitor can be assessed in a subject. The methods disclosed herein are quick, highly reproducible and relatively inexpensive. Further, the methods disclosed in the present application can be used to identify subjects suitable for a treatment with a MALT1 inhibitor, guide treatment decisions for those subjects receiving a MALT1 inhibitor therapy, and/or monitor the efficacy of an ongoing MALT1 inhibitor therapy. Further, the methods disclosed herein are not limited to monitoring the nuclear translocation of NF-κB in a tumor cell.

NF-κB nuclear translocation refers to a translocation of one or more NF-κB proteins selected from the group consisting of p50, p52, p65, Rel-B and c-Rel from the cytoplasm into the nucleus of a subject's cell. Translocation of NF-κB is a critical step in the coupling of extracellular stimuli to the transcriptional activation of specific target genes. The level of NF-κB nuclear translocation can be measured using any suitable method in view of the present disclosure, such as automated fluorescent microscopy computer-assisted image analysis technology better known as high content screening (HCS), High Content Analysis (HCS), High Content Imaging (HCl), or Image Cytometry (IC).

In some embodiments, a method of predicting a response to a MALT1 inhibitor in a subject comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; and (c) comparing the changed level of NF-kB nuclear translocation in the subject's test sample to the changed level in the control sample, wherein a decrease in the changed level of NF-kB nuclear translocation in the test sample is predictive of a positive response to the MALT1 inhibitor in the subject.

In another embodiment, a method of monitoring the efficacy of an ongoing MALT1 inhibitor therapy in a subject comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; and (c) comparing the changed level of NF-kB nuclear translocation in the subject's test sample to the changed level in the control sample, wherein a decrease in the changed level of NF-kB nuclear translocation in the test sample is indicative of efficacy of MALT1 inhibitor therapy in the subject.

In any of the methods disclosed herein, measuring the changed level of NF-kB nuclear translocation in a subject's test sample comprises:

a) obtaining a test sample of the subject;

b) contacting a first portion of the test sample with one or more stimulating agents to obtain a stimulated test sample;

c) keeping a second portion of the test sample that is not contacted with the one or more stimulating agents as an unstimulated test sample;

d) measuring a first level of NF-kB nuclear translocation from cytoplasm into nucleus of the stimulated test sample; and

e) measuring a second level of NF-kB nuclear translocation from cytoplasm into nucleus of the unstimulated test sample, wherein the cells from the stimulated sample and the unstimulated sample are of the same cell type; and

e) measuring the changed the level of NF-kB nuclear translocation in the test sample by comparing the first level of NF-kB nuclear translocation with the second level of NF-kB nuclear translocation.

In any of the methods disclosed herein, measuring the changed level of NF-kB nuclear translocation in a control sample involves similar steps as described above, and comprises:

a) obtaining a control sample of the subject;

b) contacting a first portion of the control sample with the one or more stimulating agents to obtain a stimulated control sample;

c) keeping a second portion of the control sample that is not contacted with the one or more stimulating agents as an unstimulated control sample;

c) measuring a third level of NF-kB nuclear translocation from cytoplasm into nucleus of the stimulated control sample;

d) measuring a fourth level of NF-kB nuclear translocation from cytoplasm into nucleus of the unstimulated control sample, wherein the cells from the stimulated sample and the unstimulated sample are of the same cell type; and

e) measuring the changed level of NF-kB nuclear translocation in the control sample by comparing the third level of NF-kB nuclear translocation with the fourth level of NF-kB nuclear translocation.

In some embodiments, a changed level of NF-kB nuclear translocation in the control sample is stored, and the information can be retrieved and used as a control in a method of the application. In certain embodiments, the determined changed level of NF-κB nuclear translocation in the control blood sample can be saved as part of the medical record of the subject.

Once the changed level of NF-κB nuclear translocation in the test sample and in the control sample is obtained, one can compare the changed levels between the two. By comparing the changed level of NF-kB nuclear translocation in the subject's test sample to the control sample, one skilled in the art can:
predict a response to a MALT1 inhibitor in a subject.
monitor the efficacy of an ongoing MALT1 inhibitor therapy in a subject.
treat a cancer or a MALT1-mediated disease in a subject.
design a drug regimen to treat cancer or a MALT1-mediated disease in a subject.
modify the dose and/or frequency of dosing of a MALT1 inhibitor in a subject.

In some embodiments, a decrease in the changed level of NF-kB nuclear translocation in the test sample when compared to control sample is predictive of a positive response to the MALT1 inhibitor in the subject.

In some embodiments, a decrease in the changed level of NF-kB nuclear translocation in the test sample when compared to the control sample is indicative of efficacy of MALT1 inhibitor therapy in the subject.

In some embodiments, a lower dose of MALT1 inhibitor may be administered to the subject if the test sample displays a decrease in the changed level of NF-kB nuclear translocation when compared to the control sample.

In some embodiments, a higher dose of MALT1 inhibitor may be administered to the subject if the test sample does not display a decrease in the changed level of NF-kB nuclear translocation when compared to the control sample.

In some embodiments, a second therapeutic agent may be administered to the subject if the test sample does not display a decrease in the changed level of NF-kB nuclear translocation when compared to the control sample.

In some embodiments, the dosing frequency of a MALT1 inhibitor in a subject may be reduced if the test sample displays a decrease in the changed level of NF-kB nuclear translocation when compared to the control sample.

In some embodiments, the dosing frequency of a MALT1 inhibitor may be increased if the test sample does not display a decrease in the changed level of NF-kB nuclear translocation when compared to the control sample.

In some embodiments, test sample is a subject's sample that has been exposed to a MALT1 inhibitor, and the control sample is a subject's sample that has not been exposed to a MALT1 inhibitor. Preferably, the test sample and the control sample are from the same subject. the subject.

In some embodiments, the test sample may be a subject's sample that is exposed to a MALT1 inhibitor in vitro. For example, a sample is obtained from a human subject before the subject is administered with the MALT1 inhibitor. Such a sample can be contacted with a MALT1 inhibitor in vitro to obtain a test sample. In certain embodiments, the subject's sample is contacted or incubated with a MALT1 inhibitor for about 1 to about 16 hours, about 1 to about 12 hours, about 1 to about 10 hours, or about 1 to about 8 hours. Non-limiting examples include about 2, 4, 8, 9, 10, 11, 12, 13, 14, 15 or 16 hours, preferably at 37° C., to obtain the test sample. The MALT1 inhibitor may be contacted with the sample at a concentration of about 1 to about 500 micromolar, about 1 to about 400 micromolar, about 1 to about 300 micromolar, about 1 to about 200 micromolar, or about 1 to about 100 micromolar. The test sample that is obtained may be exposed to one or more stimulating agents and the changed level of NF-κB nuclear translocation can be measured as described herein. By measuring the changed level of NF-kB nuclear translocation in the test sample, one can predict a response to a MALT1 inhibitor in a subject.

In some embodiments, the test sample may be a subject's sample that is exposed to a MALT1 inhibitor in vivo. For example, a sample is obtained from a human subject after the subject is administered with a MALT1 inhibitor. Preferably, the sample is obtained from the subject after the subject is administered with the MALT1 inhibitor at a dose from about 0.1 mg to about 3000 mg, from about 1 mg to about 1000 mg, or from about 10 mg to about 500 mg. The sample from the subject may be obtained after at least 3 hours, at least 6 hours, at last 8 hours, at least 10 hours, at least 12 hours, at least 24 hours or more after administration of the MALT1 inhibitor. The test sample that is obtained may be exposed to one or more stimulating agents and the changed level of NF-κB nuclear translocation can be measured as described herein. By measuring the changed level of NF-kB nuclear translocation in the test sample, one can predict a response to a MALT1 inhibitor in a subject.

In some embodiments, the subject's sample may be any cell or tissue. In some embodiments, the subject's sample may be a normal cell, a normal tissue, a tumor cell, a tumor tissue, or any malignant cell. In some embodiments, a subject's sample is whole blood. In some embodiments, the subject's sample may be peripheral blood mononuclear cells (PBMCs) isolated from whole blood. In some embodiments, the test sample is whole blood or PBMCs obtained from a subject who has been administered with a MALT1 inhibitor. In some embodiments, the control sample is whole blood or PBMCs obtained from a subject prior to administration with a MALT1 inhibitor. In some embodiments, the test sample and the control sample are from the same subject. In some embodiments, the test sample and the control sample are of the same cell type.

In any of the methods disclosed herein, after obtaining the subject's sample (test or control sample), the sample may be divided into parts and treated with one or more stimulating agents to obtain a stimulated test sample or a stimulated control sample. The untreated will serve as unstimulated test sample or an unstimulated control sample.

Any stimulating agent capable of activating the NF-kB pathway may be used to stimulate the subject's test sample or the control sample. In one embodiment, the stimulating agent is selected from the group consisting of a pro-inflammatory cytokine, such as an IL-1α, IL-1β, TNF-α; a bacterial toxin, such as a lipopolysaccharide (LPS), exotoxin B, phorbol myristate acetate (PMA)/ionomycin; a TLR agonist, such as CpG; an anti-CD3 antibody, anti-CD8 antibody and anti-IgM antibody, or an antigen binding fragment of the antibody, and combinations thereof. Preferably, at least one of an anti-CD3 antibody and an anti-CD28 antibody or antigen binding fragments thereof, more preferably, both the anti-CD3 antibody and the anti-CD28 antibody or antigen binding fragments thereof, are used to activate a subject's sample. In another embodiment, an anti-IgM antibody or antigen binding fragment thereof is used as a stimulating agent to activate a subject's sample.

In certain embodiments, the test sample or the control sample is contacted with one or more of the stimulating agents for about 1 to 12 hours, about 1 to 10 hours, about 1 to 9 hours, or about 1 to 8 hours. Non-limiting examples include about 1, 2, 3, 4, 5, 6, 7, 8 or 9 hours, preferably at 37° C., to obtain a stimulated test sample or a stimulated control sample.

Once a stimulated subject's sample and an unstimulated subject's sample are obtained, the level of NF-κB nuclear translocation from the cytoplasm into the nucleus of a cell in the subject's sample can be measured using any fluorescence based assay, such as flow cytometry, preferably imaging flow cytometry (IFC), luminescent analysis, chemiluminescent analysis, histochemistry, fluorescent microscopy, and the like.

In certain embodiments, NF-κB nuclear translocation from the cytoplasm into the nucleus of a subject's cell is determined using a method comprising: a) fixing the cell; b) optionally staining the cell with at least one fluorescent antibody against a surface antigen specific to the cell; c) permeabilizing the cell; d) staining the cell with a nuclear stain; e) contacting the cell with an antibody specific for a NF-κB subunit; and f) utilizing a fluorescence imaging system to determine the level of NF-κB nuclear translocation from the cytoplasm into the nucleus of the cell.

In some embodiments, the fluorescent-tagged antibody may be an antibody to a B cell surface antigen or B cell marker. In some embodiments, the fluorescent-tagged antibody may be an antibody to a T cell surface antigen or T cell marker. In certain embodiments, the fluorescent-tagged cell surface antibody is selected from the group consisting of an anti-CD3 antibody, an anti-CD4 antibody, an anti-CD5 antibody, an anti-CD8 antibody, an anti-CD19 antibody, and an anti-CD20 antibody, or an antigen binding fragment of the antibody.

In certain embodiments, the cell is permeabilized with a reagent selected from the group consisting of Triton X-100, Tween 20, saponin, digitonin, and methanol. Other reagents can also be used in view of the present disclosure.

In certain embodiments, the nuclear stain is selected from the group consisting of a DNA stain, such as 4′,6-diamidino-2-phenylindole (DAPI), propidium iodide, DRAQ5, DRAQ7 and a Hoescht stain. Other suitable nuclear strains can also be used in view of the present disclosure.

In certain embodiments, the antibody specific for the NF-κB is an antibody specific to p50, p52, p65, Rel-B or c-Rel, preferably p50.

In some embodiments, the nuclear translocation of NF-kB may be analyzed by any fluorescence-based assay in the art, such as flow cytometry, preferably imaging flow cytometry (IFC), luminescent analysis, chemiluminescent analysis, histochemistry, fluorescent microscopy, and the like.

In some embodiments, comparing the changed level of NF-κB nuclear translocation in the test sample with a changed level of NF-κB nuclear translocation in a control sample may provide information about MALT1 inhibitor efficacy in a subject. For example, a decrease in changed level of NF-κB nuclear translocation in the test sample when compared to the control sample may indicate that the MALT1 inhibitor is effective in a subject. In certain embodiments, the decrease in changed level of NF-κB nuclear translocation in the test sample when compared to the control sample is by about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.9% or more, or any range(s) in between.

In certain embodiments, the method comprises enriching or isolating PBMCs from the blood sample prior to measuring the level of NF-κB nuclear translocation into the nucleus of a PBMC. The PBMCs can be enriched or isolated from a whole blood sample using methods known in the art in view of the present disclosure. For example, PBMCs in a blood sample can be separated from red blood cells and granulocytes (neutrophils, basophils and eosinophils) by density gradient centrifugation, wherein the PBMCs remains in the low-density fraction (upper fraction), and the red blood cells and granulocytes remain in the higher density fraction (lower fraction). PBMCs can also be enriched by lysing the red blood cells in the blood sample prior to the measurement of the level of NF-κB nuclear translocation in a PBMC of interest.

PBMCs are heterogenous population of cells, and typically comprise lymphocytes in the range of 70-90%, monocytes from 10 to 20%, dendritic cells from 1-2%. The frequencies of cell types within the lymphocyte population include, e.g., 70-85% CD3+ T cells, 5-10% B cells, and 5-20% NK cells. Any PBMC present in the peripheral blood that is responsive to the one or more stimulating agents can be stimulated and analyzed in a presently described method. In certain embodiments, the PBMC is a cell selected from the group consisting of a T cell, a B cell, a natural killer cell, a monocyte, and a dendritic cell. In a preferred embodiment, the PBMC is a T cell, which can, for example, be a T cell that is CD3+, CD4+ and/or CD8+. In another embodiment, the PBMC is a B cell, which can be, for example, a CD19+ B cell.

In certain embodiments, a level of NF-κB nuclear translocation in a PBMC of a blood sample is measured without any enrichment or isolation of the PBMC. In other embodiments, a level of NF-κB nuclear translocation in a PBMC of a blood sample is measured from the PBMC after the PBMC is enriched or isolated from the blood sample.

In an exemplary embodiment, a whole blood sample from a DLBCL or CLL patient is stimulated with anti-CD3/anti-CD28 antibodies. After fixation, cell surface markers CD4, CD8 (e.g., for T cells in DLBCL patient blood), and CD19/CD20 (e.g., for B cells in CLL patient's blood) are stained with fluorescence antibodies, followed by cell permeabilization and staining with Hoechst 33342 and the p50 antibody, to identify nuclei and NF-κB, respectively. Where the MALT1 inhibitor is effective, it is found that p50 nuclear translocation is dramatically blocked in the stimulated T cells obtained from DLBCL patients, as well as in malignant B cells from CLL patients.

In some embodiments, the efficacy of MALT1 inhibitor in a subject can also be monitored by measuring the expression of CD69 marker on a T cell. It is known that activation NF-kB pathway results in expression of CD69 in T cells. In some embodiments, the methods disclosed herein can be used to monitor the expression of CD69 in T cells in a subject administered with a MALT1 inhibitor. In some embodiments, the method comprises: a) measuring a first CD69 expression level from a T cell in the stimulated test sample; b) measuring a second CD69 expression level from a T cell in the unstimulated test sample; c) comparing the first CD69 expression level with the second CD69 expression level to thereby determine a changed level of CD69 expression in the test sample; and d) comparing the changed level of CD69 expression in the test sample with a control sample.

In some embodiments, a changed level of CD69 expression in a control sample is measured by a method comprising: a) measuring a third CD69 expression level from a T cell in the stimulated control sample; b) measuring a fourth CD69 expression level from a T cell in the unstimulated control sample; and c) comparing the third CD69 expression level with the fourth CD69 expression level to thereby determine the changed level of CD69 expression in a control sample. The changed level of CD69 expression in a control sample can be stored, and the stored information can be retrieved and used as a control in a method of the application.

In some embodiments, nuclear translocation of a MALT1-independent marker can be monitored to confirm the activation of the NF-κB pathway in a sample. Examples of the MALT1-independent marker include, but are not limited to, nuclear factor of activated T-cells (NFAT) and STAT3. NFAT is a family of transcription factors involved in regulating the immune response. The canonical NFAT pathway is calcium-dependent and upon activation, NFAT is dephosphorylated by the phosphatase, calcineurin. This results in its translocation from the cytoplasm to the nucleus and transcription of downstream target genes that include the cytokines IL-2, IL-10, and IFNγ. A changed level of a MALT1-independent marker in a subject's sample can be determined, for example, by measuring a first level and a second level of the MALT1-independent marker in the stimulated sample and unstimulated sample, respectively, in the presence or absence of the MALT1 inhibitor, and comparing the first level with the second level. In certain embodiments, the changed level of a MALT1-independent marker can be saved as part of the medical record of the subject and it can be used as a control in a method according to an embodiment of the application.

Also disclosed herein are methods to treat a subject. In some embodiments, a method of treating a cancer or a MALT1-mediated disease in a subject in need thereof comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; (c) comparing the changed level of NF-kB nuclear translocation in the subject's test sample to the changed level in the control sample; and (d) administering a lower dose of MALT1 inhibitor to the subject if the test sample displays a decrease in the changed level of NF-kB nuclear translocation, and administering a higher dose of MALT1 inhibitor to the subject if the test sample does not display a decrease in the changed level of NF-kB nuclear translocation.

In another embodiment, a method of treating a cancer or a MALT1-mediated disease in a subject comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; (c) comparing the changed level of NF-kB nuclear translocation in the subject's test sample to the changed level in the control sample; and (d) administering an effective amount of MALT1 inhibitor to the subject if the test sample displays a decrease in the changed level of NF-kB nuclear translocation.

In some embodiments, a method of treating a cancer or a MALT1-mediated disease in a subject in need thereof comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; (c) comparing the changed level of NF-kB nuclear translocation in the subject's test sample to the changed level in the control sample; and (d) continuing the treatment method if the test sample displays a decrease in the changed level of NF-kB nuclear translocation, and stopping the treatment method if the test sample does not display a decrease in the changed level of NF-kB nuclear translocation.

In some embodiments, a method of designing a drug regimen to treat cancer or a MALT1-mediated disease in a subject comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; (c) comparing the changed level of NF-kB nuclear translocation in the subject's test sample to the changed level in the control sample; and (d) administering a second therapeutic agent to the subject if the test sample does not display a decrease in the changed level of NF-kB nuclear translocation.

In some embodiments, a method of modifying the dose and/or frequency of dosing of a MALT1 inhibitor in a subject suffering from cancer or a MALT1-mediated disease comprises: (a) measuring the changed level of NF-kB nuclear translocation in a subject's test sample that has been previously exposed to a MALT1 inhibitor; (b) measuring the changed level of NF-kB nuclear translocation in a subject's control sample that has not been previously exposed to a MALT1 inhibitor; (c) comparing the changed level of NF-kB nuclear translocation in the subject's test sample to the changed level of the control sample; and (d) reducing the dosing frequency of a MALT1 inhibitor if the test sample displays a decrease in the changed level of NF-kB nuclear translocation, and increasing the dosing frequency of a MALT1 inhibitor if the test sample does not display a decrease in the changed level of NF-kB nuclear translocation.

In some embodiments, MALT1-mediated disease is cancer. In certain embodiments, the cancer is selected from the group consisting of a lymphoma, a leukemia, a carcinoma, and a sarcoma. The cancer can, for example, be selected from the group consisting of non-Hodgkin's lymphoma, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), lymphoblastic T cell leukemia, chronic myelogenous leukemia (CVL), small lymphocytic lymphoma (SLL), Waldenstrom macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocytic leukemia, promyelocytic leukemia, erytholeukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

In one embodiment, the human subject is in need of a treatment for lymphoma, such as a Hodgkin lymphoma or a non-Hodgkin lymphoma (NHL), preferably a diffuse large B-cell lymphoma (DLBCL), more preferably an activated B-cell-like (ABC) subtype of DLBCL. In another embodiment, the human subject is in need of a treatment for leukemia, such as an acute lymphocytic leukemia, a chronic lymphocytic leukemia (CLL), an acute myeloid leukemia, or a chronic myeloid leukemia, preferably the CLL.

In yet another embodiment, the MALT1-mediated disease is an immunological disease including, but not limited to, an autoimmune and inflammatory disorder, e.g. arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's disease, uveitis, myasthenia gravis, Grave's disease, Hashimoto thyroiditis, Sjoergen's syndrome, a blistering disorder, antibody-mediated vasculitis syndromes, immune-complex vasculitides, an allergic disorder, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

In some embodiments, the method of treating a cancer or a MALT1-mediated disease in a subject comprises administering a lower dose of MALT1 inhibitor to the subject if the test sample displays a decrease in the changed level of NF-kB nuclear translocation. In some embodiments, the subject may be administered with a lower dose of MALT1 inhibitor selected from about 1 mg, about 10 mg, about 50 mg, about 100 mg, about 150 mg, about 200 mg, or about 250 mg.

In some embodiments, the method of treating a cancer or a MALT1-mediated disease in a subject comprises administering a higher dose of MALT1 inhibitor to the subject if the test sample does not display a decrease in the changed level of NF-kB nuclear translocation. In some embodiments, the subject may be administered with a higher dose of MALT1 inhibitor selected from about 500 mg, about 1000 mg, or about 3000 mg.

In some embodiments, the method of treating a cancer or a MALT1-mediated disease in a subject comprises administering an effective amount of MALT1 inhibitor to the subject if the test sample displays a decrease in the changed level of NF-kB nuclear translocation. In some embodiments, the effective amount of MALT1 inhibitor is from about 0.1 mg to about 3000 mg, from about 1 mg to about 1000 mg, or from about 10 mg to about 500 mg.

In some embodiments, a method of designing a drug regimen to treat cancer or a MALT1-mediated disease in a subject comprises administering a second therapeutic agent to the subject if the test sample does not display a decrease in the changed level of NF-kB nuclear translocation. For example, the second therapeutic agent that may be administered is selected from BTK (Bruton's tyrosine kinase) inhibitors such as ibrutinib, SYK inhibitors, PKC inhibitors, PI3K pathway inhibitors, BCL family inhibitors, JAK inhibitors, PIM kinase inhibitors, rituximab or other B cell antigen-binding antibodies, as well as immune cell redirection agents (e.g. blinatumomab or CAR T-cells) and immunomodulatory agents such as daratumumab, anti-PD1 antibodies, and anti-PD-L1 antibodies.

In some embodiments, a method of modifying the dose and/or frequency of dosing of a MALT1 inhibitor in a subject suffering from cancer or a MALT1-mediated disease comprises decreasing the dosing frequency of a MALT1 inhibitor if the test sample displays a decrease in the changed level of NF-kB nuclear translocation. In some embodiments, the subject may be administered with a lower dosing frequency of MALT1 inhibitor, such as once daily. The effective amount of MALT1 inhibitor that may be administered may be from about 1 mg to about 1000 mg.

In some embodiments, a method of modifying the dose and/or frequency of dosing of a MALT1 inhibitor in a subject suffering from cancer or a MALT1-mediated disease comprises increasing the dosing frequency of a MALT1 inhibitor if the test sample does not display a decrease in the changed level of NF-kB nuclear translocation. In some embodiments, the subject may be administered with a higher dosing frequency of MALT1 inhibitor, such as twice daily or thrice daily or four times per day. The effective amount of MALT1 inhibitor that may be administered may be from about 1 mg to about 1000 mg.

In some embodiments, the compositions of MALT1 inhibitors disclosed herein may be administered to a subject by a variety of routes such as subcutaneous, topical, oral and intramuscular. Administration of the compositions may be accomplished orally or parenterally. Methods of parenteral delivery include topical, intra-arterial (directly to the tissue), intramuscular, subcutaneous, intramedullary, intrathecal, intraventricular, intravenous, intraperitoneal, or intranasal administration.

In certain embodiments, the method can further comprises determining whether the subject has a mutation in a CD79B gene. In certain embodiments, the method further comprises determining whether the subject has a mutation in a CARD11 gene. Methods of determining whether the subject has a mutation in a CD79B or CARD11 gene are known in the art. By way of a non-limiting example, the gene (e.g., CD79B or CARD11) could be sequenced and compared with a wild-type version of the gene.

Embodiments of the application also include a MALT1 inhibitor for use in treating a MALT1-mediated disease in a subject in need thereof, wherein it is determined that the MALT1 inhibitor is efficacious against the MALT1-mediated disease in the subject using a method according to an embodiment of the application.

The invention relates to a MALT1 inhibitor for use in a method as described in any one of the other embodiments.

The invention relates to a MALT1 inhibitor for use in a method of treating a MALT1-mediated disease as described in any one of the other embodiments.

The invention relates to a MALT1 inhibitor for use in treating a MAL T1-mediated disease as described in any one of the other embodiments.

The invention relates to a MALT1 inhibitor for use in a treatment of a MALT1-mediated disease as described in any one of the other embodiments.

It will be appreciated that a MALT1 inhibitor for use in a method of diagnosis in vivo provided herein may encompass a MALT1 inhibitor for use in a method of diagnosis practised on the human or animal body.

### Compositions

Also disclosed herein are compositions of MALT1 inhibitor. In some embodiments, a MALT1 inhibitor is a compound of Formula (I)

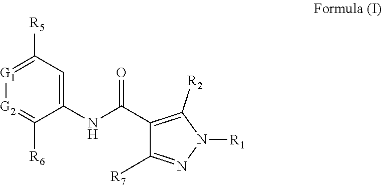

wherein
R₁ is selected from the group consisting of
i) naphthalen-1-yl, optionally substituted with a fluoro or amino substituent; and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, cyclopropyl, methoxymethyl, difluoromethyl, 1,1-difluoroethyl, hydroxymethyl, 1-hydroxyethyl, 1-ethoxyethyl, hydroxy, methoxy, ethoxy, fluoro, chloro, bromo, methylthio, cyano, amino, methylamino, dimethylamino, 4-oxotetrahydrofuran-2-yl, 5-oxopyrrolidin-2-yl, 1,4-dioxanyl, aminocarbonyl, methylcarbonyl, methylaminocarbonyl, oxo, 1-(t-butoxycarbonyl)azetidin-2-yl, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
R₂ is selected from the group consisting of C₁₋₄alkyl, 1-methoxy-ethyl, difluoromethyl, fluoro, chloro, bromo, cyano, and trifluoromethyl;
G₁ is N or C(R₄);
G₂ is N or C(R₃); such that only one of G₁ and G₂ are N in any instance;
R₃ is independently selected from the group consisting of trifluoromethyl, cyano, C₁₋₄alkyl, fluoro, chloro, bromo, methylcarbonyl, methylthio, methylsulfinyl, and methanesulfonyl; or, when G₁ is N, R₃ is further selected from C₁₋₄alkoxycarbonyl;
R₄ is selected from the group consisting of
i) hydrogen, when G₂ is N;
ii) C₁₋₄alkoxy;
iii) cyano;
iv) cyclopropyloxy;
v) a heteroaryl selected from the group consisting of triazolyl, oxazolyl, isoxazolyl, pyrazolyl, pyrrolyl, thiazolyl, tetrazolyl, oxadiazolyl, imidazolyl, 2-amino-pyrimidin-4-yl, 2H-[1,2,3]triazolo[4,5-c]pyridin-2-yl, 2H-[1,2,3]triazolo[4,5-b]pyridin-2-yl, 3H-[1,2,3]triazolo[4,5-b]pyridin-3-yl, 1H-[1,2,3]triazolo[4,5-c]pyridin-1-yl, wherein the heteroaryl is optionally substituted with one or two substituents independently selected from oxo, C₁₋₄alkyl, carboxy, methoxycarbonyl, aminocarbonyl, hydroxymethyl, aminomethyl, (dimethylamino)methyl, amino, methoxymethyl, trifluoromethyl, amino(C₂₋₄alkyl)amino, or cyano;
vi) 1-methyl-piperidin-4-yloxy;
vii) 4-methyl-piperazin-1-ylcarbonyl;
viii) (4-aminobutyl)aminocarbonyl;
ix) (4-amino)butoxy;
x) 4-(4-aminobutyl)-piperazin-1-ylcarbonyl;
xi) methoxycarbonyl;
xii) 5-chloro-6-(methoxycarbonyl)pyridin-3-ylaminocarbonyl;
xiii) 1,1-dioxo-isothiazolidin-2-yl;
xiv) 3-methyl-2-oxo-2,3-dihydro-1H-imidazol-1-yl;
xv) 2-oxopyrrolidin-1-yl;
xvi) (E)-(4-aminobut-1-en-1-yl-aminocarbonyl;
xvii) difluoromethoxy; and
xviii) morpholin-4-ylcarbonyl;
R₅ is independently selected from the group consisting of hydrogen, chloro, fluoro, bromo, methoxy, methylsulfonyl, cyano, C₁₋₄alkyl, ethynyl, morpholin-4-yl, trifluoromethyl, hydroxyethyl, methylcarbonyl, methylsulfinyl, 3-hydroxy-pyrrolidin-1-yl, pyrrolidin-2-yl, 3-hydroxyazetidinyl, azetidin-3-yl, azetidin-2-yl, methylthio, and 1,1-difluoroethyl;
or R₄ and R₅ can be taken together to form 8-chloro-4-methyl-3-oxo-3,4-dihydro-2H-benzo[b][1,4]oxazin-6-yl, 8-chloro-3-oxo-3,4-dihydro-2H-benzo[b][1,4]oxazin-6-yl, 2-methyl-1-oxo-1,2,3,4-tetrahydroisoquinolin-7-yl, 4-methyl-3-oxo-3,4-dihydro-2H-benzo[b][1,4]oxazin-6-yl, 3-oxo-3,4-dihydro-2H-benzo[b][1,4]oxazin-6-yl, 1-methyl-1H-pyrazolo[3,4-b]pyridin-5-yl, 1H-pyrazolo[3,4-b]pyridin-5-yl, 2,3-dihydro-[1,4]dioxino[2,3-b]pyridin-5-yl, 1,3-dioxolo[4,5]pyridine-5-yl, 1-oxo-1,3-dihydroisobenzofuran-5-yl, 2,2-dimethylbenzo[d][1,3]dioxol-5-yl, 2,3-dihydrobenzo[b][1,4]dioxin-6-yl, 1-oxoisoindolin-5-yl, or 2-methyl-1-oxoisoindolin-5-yl, 1H-indazol-5-yl;
R₆ is hydrogen, C₁₋₄alkyl, fluoro, 2-methoxy-ethoxy, chloro, cyano, or trifluoromethyl;
R₇ is hydrogen or fluoro;
provided that a compound of Formula (I) is other than
a compound wherein R₁ is isoquinolin-8-yl, R₂ is trifluoromethyl, G₁ is C(R₄) wherein R₄ is 2H-1,2,3-triazol-2-yl, G₂ is N, and R₅ is hydrogen;
a compound wherein R₁ is isoquinolin-8-yl, R₂ is trifluoromethyl, G₁ is C(R₄) wherein R₄ is 1H-imidazol-1-yl, G₂ is N, and R₅ is chloro;
a compound wherein R₁ is isoquinolin-8-yl, R₂ is trifluoromethyl, G₁ is C(R₄) wherein R₄ is 1H-1,2,3-triazol-1-yl, G₂ is N, and R₅ is hydrogen;
a compound wherein R₁ is isoquinolin-8-yl, R₂ is trifluoromethyl, G₁ is C(R₄) wherein R₄ is hydrogen, G₂ is N, and R₅ is fluoro;
or an enantiomer, diastereomer, solvate, or pharmaceutically acceptable salt form thereof.

In certain embodiments, a MALT1 inhibitor useful for the invention, as well as related information such as its structure, production, biological activities, therapeutic applications, administration or delivery, etc., is described in US20180170909 and WO2018/119036, the content of which is incorporated herein by reference in its entirety.

In some embodiments, a MALT1 inhibitor is “Compound A” and refers to a compound of 1-(1-oxo-1,2 dihydroisoquinolin-5-yl)-5 (trifluoromethyl)-N-[2 (trifluoromethyl)pyridin-4 yl]-1H-pyrazole-4 carboxamide, which has the structure of Formula (II):

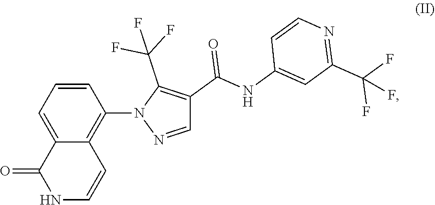 [SMILES: O=C(Nc1ccnc(C(F)(F)F)c1)c1cnn(-c2cccc3c(=O)[nH]ccc23)c1C(F)(F)F]

or a solvate, a tautomer, or a pharmaceutically acceptable salt thereof. In certain embodiment, Compound A is a monohydrate form of the compound of formula (II).

Compound A can be prepared, for example, as described in Example 158 of US20180170909, which is incorporated herein by reference in its entirety. The procedure of Example 158 has been determined as providing a hydrate form of the compound of Formula (II).

Compound A is an orally bioavailable, potent, and selective MALT1 inhibitor that binds to an allosteric site with a mixed-type mechanism. In nonclinical studies, Compound A has been shown to inhibit growth of cluster of differentiation (CD)79b-mutant DLBCL and ibrutinib-resistant DLBCL cell lines harboring Bruton tyrosine kinase (BTK) C481S or caspase recruitment domain-containing protein 11 (CARD11) mutations in vitro, and has shown efficacy in a CD79b and CARD11-mutant ABC-DLBCL xenograft models in vivo. At a single dose of either 1 M or 10 M, Compound A did not show significant binding inhibition of proteases, caspases, protein kinases, and G-protein-coupled receptors.

Compound A can exist as a solvate. A “solvate” can be a solvate with water (i.e., a hydrate) or with a common organic solvent. The use of pharmaceutically acceptable solvates, said solvates including hydrates, and said hydrates including mono-hydrates, is considered to be within the scope of the invention.

Compound A can be formulated in an amorphous form or dissolved state, for example and without limitation, Compound A can be formulated in an amorphous form with a polyethylene glycol (PEG) polymer.

A person of ordinary skill in the art would recognize that Compound A can exist as tautomers. It is understood that all tautomeric forms are encompassed by a structure where one possible tautomeric arrangement of the groups of the compound is described, even if not specifically indicated.

For example, it is understood that:

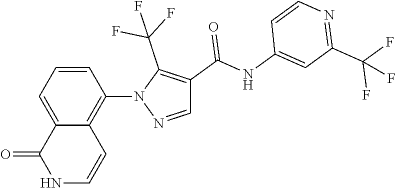 [SMILES: O=C(Nc1ccnc(C(F)(F)F)c1)c1cnn(-c2cccc3c(=O)[nH]ccc23)c1C(F)(F)F]

also encompasses by the following structure:

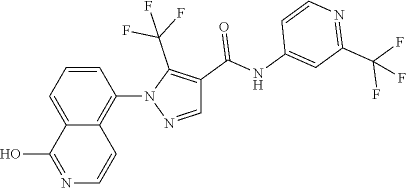 [SMILES: O=C(Nc1ccnc(C(F)(F)F)c1)c1cnn(-c2cccc3c(O)nccc23)c1C(F)(F)F]

Any convenient tautomeric arrangement can be utilized in describing the compounds.

A MALT1 inhibitor can be administered to a subject in any suitable pharmaceutical compositions. It can be admixed with any suitable binder(s), lubricant(s), suspending agent(s), coating agent(s), solubilizing agent(s), and combinations thereof. For example, solid oral dosage forms such as, tablets or capsules, containing the compounds of the present invention can be administered in at least one dosage form at a time, as appropriate. It is also possible to administer the compounds in sustained release formulations. Additional oral forms in which the present inventive compounds can be administered include elixirs, solutions, syrups, and suspensions; each optionally containing flavoring agents and coloring agents. Alternatively, a MALT1 inhibitor can be administered by inhalation (intratracheal or intranasal) or in the form of a suppository or pessary, or they can be applied topically in the form of a lotion, solution, cream, ointment or dusting powder. For example, they can be incorporated into a cream comprising, consisting of, and/or consisting essentially of an aqueous emulsion of polyethylene glycols or liquid paraffin. They can also be incorporated, at a concentration of between about 1% and about 10% by weight of the cream, into an ointment comprising, consisting of, and/or consisting essentially of a wax or soft paraffin base together with any stabilizers and preservatives as can be required. An alternative means of administration includes transdermal administration by using a skin or transdermal patch.

The pharmaceutical compositions of MALT1 inhibitor (as well as the compounds of the present invention alone) can also be injected parenterally, for example, intracavernosally, intravenously, intramuscularly, subcutaneously, intradermally, or intrathecally. In this case, the compositions will also include at least one of a suitable carrier, a suitable excipient, and a suitable diluent.

For parenteral administration, the pharmaceutical compositions of the present invention are best used in the form of a sterile aqueous solution that can contain other substances, for example, enough salts and monosaccharides to make the solution isotonic with blood. For buccal or sublingual administration, the pharmaceutical compositions of the present invention can be administered in the form of tablets or lozenges, which can be formulated in a conventional manner.

By way of further example, pharmaceutical compositions containing a MALT1 inhibitor, such as a compound of Formula (I) or (II), as the active ingredient can be prepared by mixing the compound(s) with a pharmaceutically acceptable carrier, a pharmaceutically acceptable diluent, and/or a pharmaceutically acceptable excipient according to conventional pharmaceutical compounding techniques. The carrier, excipient, and diluent can take a wide variety of forms depending upon the desired route of administration (e.g., oral, parenteral, etc.). Thus, for liquid oral preparations such as, suspensions, syrups, elixirs and solutions, suitable carriers, excipients and diluents include water, glycols, oils, alcohols, flavoring agents, preservatives, stabilizers, coloring agents and the like; for solid oral preparations such as, powders, capsules, and tablets, suitable carriers, excipients and diluents include starches, sugars, diluents, granulating agents, lubricants, binders, disintegrating agents and the like. Solid oral preparations also can be optionally coated with substances such as, sugars, or be enterically coated so as to modulate the major site of absorption and disintegration. For parenteral administration, the carrier, excipient and diluent will usually include sterile water, and other ingredients can be added to increase solubility and preservation of the composition. Injectable suspensions or solutions can also be prepared utilizing aqueous carriers along with appropriate additives such as, solubilizers and preservatives.

A therapeutically effective amount of a compound of Formula (I) or (II) or a pharmaceutical composition thereof includes a dose range from about 0.1 mg to about 3000 mg, or any particular amount or range therein, in particular from about 1 mg to about 1000 mg, or any particular amount or range therein, or, more particularly, from about 10 mg to about 500 mg, or any particular amount or range therein, of active ingredient in a regimen of about 1 to about (4×) per day for an average (70 kg) human; although, it is apparent to one skilled in the art that the therapeutically effective amount for a compound of Formula (I) will vary as will the diseases, syndromes, conditions, and disorders being treated.

For oral administration, a pharmaceutical composition is preferably provided in the form of tablets containing about 1.0, about 10, about 50, about 100, about 150, about 200, about 250, and about 500 milligrams of a compound of Formula (I).

An embodiment of the present invention is directed to a pharmaceutical composition for oral administration, comprising a compound of Formula (I) in an amount of from about 25 mg to about 500 mg.

Advantageously, a compound of Formula (I) can be administered in a single daily dose, or the total daily dosage can be administered in divided doses of two, three and (4×) daily.

Optimal dosages of a compound of Formula (I) to be administered can be determined and will vary with the particular compound used, the mode of administration, the strength of the preparation, and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

Also disclosed herein are kits for measuring the nuclear translocation of NF-kB. In some embodiments, the kit comprises:
one or more agents for stimulating a PBMC in a blood sample;
one or more agents for fixing the PBMC;
one or more labeled antibodies against a surface antigen specific to the PBMC;
one or more agents for permeabilizing the PBMC;
one or more agents for staining the nuclear of the PBMC; and
one or more labeled antibodies specific for NF-κB.

### EXAMPLES

### Example 1: T Cell Activation and PBMCs Isolation for NFκB Nuclear Translocation Assays

Samples of whole blood (40 mL) were obtained from lymphoma donors in four-10 mL Heparin tubes. The samples were shipped overnight at ambient temperature from Conversant Bio (Huntsville, Ala.) collection sites. However, subsequent evidence in the lab suggested that shipping at 4° C. may better preserve the responsiveness of the cells.

Each of 6.5 mL of the whole blood sample was transferred to two 50 mL conical tubes (Corning, cat. #430290; Corning, N.Y.) and mixed 1:1 with room-temperature 1640 Roswell Park Memorial Institute (RPMI) with 25 mM HEPES (Life Technologies, cat. #72400-047), supplemented with 10% HI Fetal Bowine Serum (FBS) (Life Technologies, cat. #16140-071; Carlsbad, Calif.). One of the 50 mL sample containing conical tubes was treated with 200 μM Compound A (200 mM stock; 1000×) and the other 50 mL conical tube was treated with an equivalent volume of vehicle control DMSO (Life Technologies, cat. #L34957). Both tubes were mixed well. The treated blood mixture was transferred at 3 mL per well to a 6-well polystyrene culture plate (Falcon, cat. #353046) and incubated overnight in a humidified incubator at 37° C. with 5% CO₂.

Following overnight incubation, anti-CD3 (UCHT1 clone; BioLegend, cat. #300465; San Diego, Calif.) and anti-CD28 (ANC28.1 clone; Ancell, cat. #177-024; British Columbia, Canada) antibodies were added at a final concentration of 1 μg/mL each (1 mg/mL stock; 1000×) to the wells that require T cell stimulation. The solution was mixed by pipetting with a 1 mL pipette. The plates were incubated in a humidified incubator at 37° C. with 5% CO₂ for an additional 6 hours.

After incubation, the blood mixtures were collected into 50 mL conical tubes and centrifuged at 1,500 rpm for 5 mins at 4° C. Carefully, 1-2 mL of supernatants (mixture of plasma and culture media) were collected and frozen in small aliquots at −80° C.

The peripheral blood mononuclear cells (PBMCs) in the remaining samples were isolated by density gradient centrifugation. More specifically, sterile phosphate buffer saline (PBS, Ca++/Mg++ free; ThermoFisher Scientific, cat. #14190-144; Waltham, Mass.) (10 mL) was added to the remaining samples and mixed to reconstitute the blood cells. Then 17 mL of Ficoll Paque (GE Healthcare, cat. #17-1440-03; Chicago, Ill.) was added to the lower chamber of the 50 mL SepMate tubes (STEMCELL Technologies; cat. #85450; Vancouver, Canada) and slowly overlayed with the sample mixture. The SepMate tubes were centrifuged at 2000 rpm for 10 mins at 4° C., with brakes on. The supernatant containing the PBMCs was added to a new 50 mL conical tube and washed once with PBS. The supernatant was centrifuged at 1,500 rpm for 5 mins at 4° C.

If the PBMC pellet contained large amounts of red blood cells (RBCs), the pellets were reconstituted in 10 mL of 1×RBC lysis buffer (Invitrogen, cat. #00-4300-54; Carlsbad, Calif.) and incubated for 3 minutes before centrifuging at 1,500 rpm for 5 mins at 4° C. The supernatants were aspirated, making sure the cell pellet was intact. The pellets were reconstituted in 1 mL of freezing media (Life Technologies, cat. #12648-010), frozen, and stored in liquid nitrogen for later analysis of NF-κB nuclear translocation. Alternatively, the pellets were reconstituted in PBS and subject to NF-κB nuclear translocation analysis directly.

### Example 2: NF-kB Nuclear Translocation in T or B Cells by Imaging Flow Cytometry

Frozen or fresh cells treated with the experimental conditions were obtained. For example, T cells in blood samples could be activated and the PBMCs containing the activated T cells could be isolated using the method described in Example 1. Alternatively, PBMCs in blood samples could be activated and subject to the imaging flow cytometry analysis directly without isolation. If the samples were whole blood, a minimum of 1 mL of blood was used for each test in this experiment. However, less than 1 mL whole blood can also be used in the assay. If the samples were frozen, the samples were thawed at 37° C. and gently washed in room-temperature PBS (Life Technologies, cat. #14190-136) by centrifugation at 1350 rpm for 5 minutes.

The cells were stained for surface markers, such as CD4 (Miltenyi, cat. #130-092-373; Bergisch Gladbach, Germany) and CD8 (BioLegend, cat. #301050) (for T cells) or CD19 (BioLegend, cat. #302206) (for B cells) as well as viability dye (Life Technologies, cat. #L10119) in FACS stain buffer (BD, cat. #554657) at room temperature for 15 minutes. The cells were centrifuged at 1350 rpm for 5 minutes at room temperature, the supernatants were discarded, and the cells were washed with FACS buffer. The cells were centrifuged again at 1350 rpm for 5 minutes at room temperature, and the supernatants were discarded.

The cells were fixed in CytoFix buffer, 4.2% Formaldehyde (BD, cat. #554655; Franklin Lakes, N.J.) for 15 minutes at room temperature in the dark. The fixed cells were centrifuged at 1350 rpm for 3 minutes, the supernatants were discarded, and the fixed cells were washed with FACS buffer. The fixed cells were centrifuged again at 1350 rpm for 5 minutes at room temperature, and the supernatants were discarded.

The cells were permeabilized in 0.1% Triton® X-100 solution (VWR, cat. #0694-1L; Radnor, Pa.) in room-temperature PBS. The samples were incubated at room temperature and covered from light for 5 minutes. The cells were centrifuged at 1800 rpm for 5 minutes at 4° C. The pellets were inspected, and the supernatants were discarded.

The cells were blocked with cold FACS buffer with 1.5% BSA (Fraction V, 7.5% solution; Life Technologies, cat. #15260-037) for 15 minutes. The cells were then centrifuged at 1800 rpm for 5 minutes at 4° C., and the supernatants were discarded.

The staining solution was prepared by diluting Hoechst 33342 (Thermo Scientific, cat. #62249) to 10 nM and the p50 antibody (Clone 2J10D7; Novus, cat. #NB100-56583C) at 50 μg/mL in FACS buffer. The cells were incubated in the staining solution for 30 minutes at room temperature in the dark. The cells were washed by centrifugation at 1350 rpm for 5 minutes at room temperature, the supernatants were discarded, and the pellets were reconstituted in FACS buffer. The wash step was repeated twice, and the cells were resuspended in PBS at a final concentration of 5-20×10⁶ cell/mL in 25 μL of PBS.

The samples were imaged on an AMNIS® IMAGESTREAM® X Mark II imaging flow cytometer (MilliporeSigma, Burlington, Mass.) immediately, using 60× magnification, and the data was analyzed in IDEAS software using an internalization module, e.g., to evaluate frequency of CD4+ and CD8+ T cells or CLL cells with nuclear enrichment of p50.

The NF-κB nuclear translocation can also be measured by nuclear enrichment of p65 (the other subunit of NF-κB) with a p65 antibody using a method similar to that described above for the measurement of the nuclear enrichment of p65.

### Example 3: CD69 Expression Analysis on T Cells from Peripheral Whole Blood Samples of Normal and NHL Patients

Peripheral whole blood from normal and NHL donors was treated with 200 μM Compound A or left untreated and incubated at 37° C. overnight. The next day, blood was treated with anti-CD3 and anti-CD28 stimulatory antibodies for 6 hours as described in Example 1 or left untreated. After treatment with the stimulatory antibodies, the red blood cells were lysed using multi-species lysis buffer, and the white blood cells were stained with anti-CD4 and anti-CD8 antibodies to label T cells and an anti-CD69 antibody to measure early T cell activation. Frequency of CD69-positive T cells (CD4+ and CD8+) was measured by IFC.

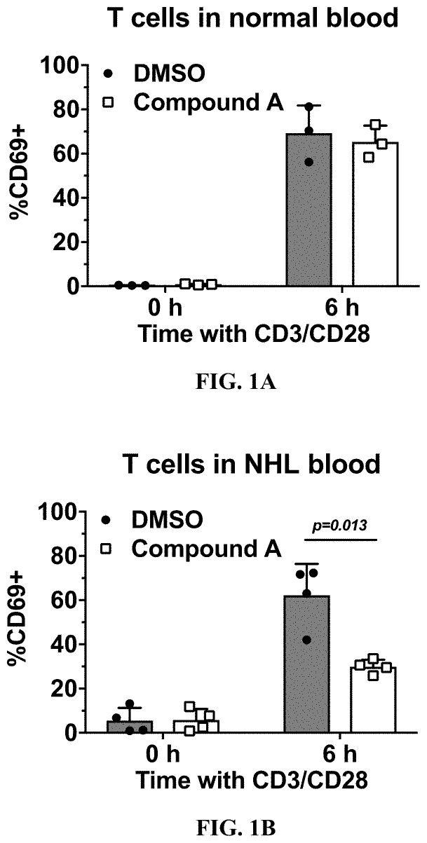

As shown in FIG. 1A, incubating the normal blood sample with anti-CD3 and anti-CD28 stimulatory antibodies resulted in an increased surface expression of CD69 on CD4+ and CD8+ T cells, and such increase was not affected by the treatment with Compound A. However, as shown in FIG. 1B, with the NHL blood sample, treatment with Compound A significantly inhibited the surface expression of CD69 on T cells activated by the anti-CD3 and anti-CD28 stimulatory antibodies.

### Example 4: NF-kB Nuclear Translocation in T Cells from Peripheral Whole Blood Samples of NHL Patients

A peripheral whole blood sample from NHL donors was mixed with equal volume of room-temperature RPMI 1640 with 25 mM HEPES, supplemented with 10% heat-inactivated fetal bovine serum, aliquoted into 96 well U-bottom plate and treated with serial dilutions of Compound A. The blood mixture samples were then incubated at 37° C. overnight. The next day, the mixture samples were treated with anti-CD3 and anti-CD28 stimulatory antibodies following a procedure as that described in Example 1. After 6-hour incubation, red blood cells in the samples were lysed using multi-species lysis buffer. White blood cells were fixed using CytoFix buffer and permeabilized using 0.1% Triton X-100 solution. Cells were then stained with anti-CD4 and anti-CD8 antibodies to label T cells, Hoechst 33342 to label nuclei and anti-p105/p50 (clone 2J10D7, Novus Biologicals) to label the NF-κB subunit. Samples were analyzed on ImageStreamX to evaluate NF-κB nuclear localization in the CD4 and the CD8 positive T cells. Relative translocation was demonstrated, and the corresponding IC₅₀ values were calculated using a nonlinear regression fit.

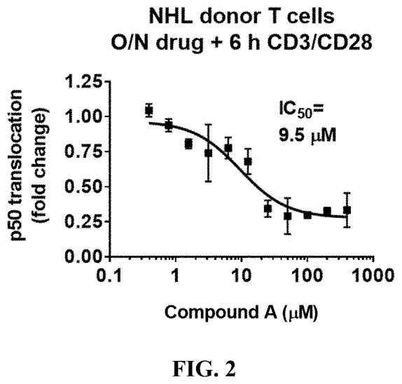

It was shown that anti-CD3 (UCHT1) and anti-CD28 (ANC28.1) activated T cells in the blood samples from normal and lymphoma subjects (data not shown). The TCR and CD28 pathways involve MALT1 signaling. As shown in FIG. 2, Compound A completely blocked canonical NF-κB signaling, e.g., NFκB nuclear translocation, in T cells activated by CD3/CD28 stimulation in NHL blood sample in a dosage dependent manner (IC50˜9.5 μM).

### Example 5: NF-κB Nuclear Translocation in B Cells from Peripheral Whole Blood Samples of Chronic Lymphocytic Leukemia (CLL) Patients

Frozen peripheral blood mononuclear cells (PBMCs) from CLL donors were thawed and incubated with the indicated concentrations of Compound A for 6 hours at 37° C. Cells were then treated with the stimulatory soluble anti-IgM F(ab′)2 fragment anti-IgM (Jackson ImmunoResearch cat. 109-006-129) or left untreated for 30 minutes. Alternatively, B cells can also be activated by beads coated with anti-IgM antibody. After the stimulation, PBMCs were fixed using CytoFix buffer and permeabilized using 0.1% Triton X-100 solution. Cells were then stained with anti-CD19 to label B (CLL) cells, Hoechst 33342 to label nuclei, and anti-p105/p50 to label NF-κB subunit. Samples were analyzed on ImageStreamX to evaluate NF-κB nuclear localization in the CD19-positive cells. Translocation indices (similarity scores) for p105/p50 staining and Hoechst 33342 staining were demonstrated. Statistical significance was determined using Student's t-test in Microsoft Excel.

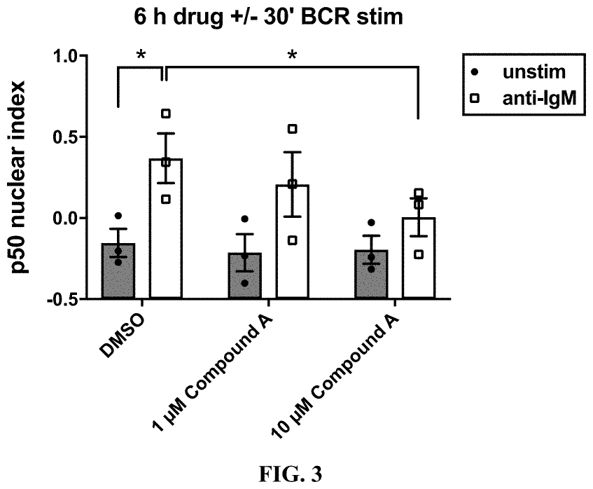

As shown in FIG. 3, stimulation of the PBMCs with anti-IgM resulted in activated B cells in the CLL blood sample, which exhibited nuclear enrichment of p50, e.g., activated B cells had increased NF-κB nuclear translocation from the cytoplasm to the nucleus. It was also shown that Compound A inhibited the NF-κB nuclear translocation in the activated B cells.

### Example 6: NF-κB Nuclear Translocation in B Cells and T Cells from Whole Blood Samples of CLL Patients

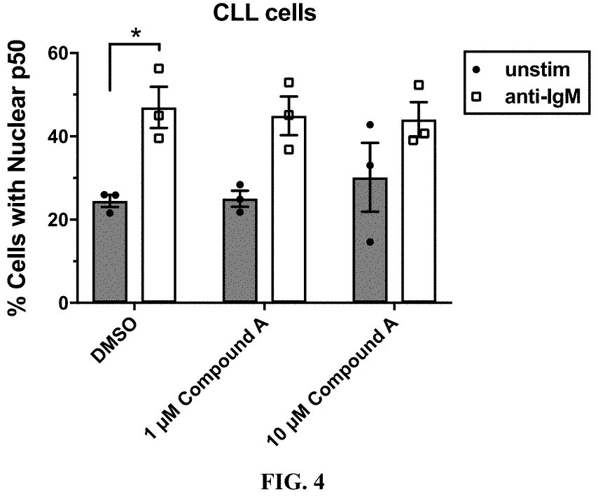

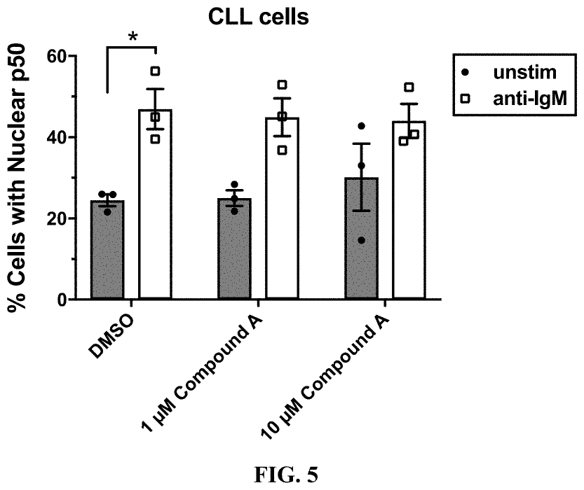

Frozen PBMC from CLL donors were thawed and incubated with the indicated concentrations of Compound A at 37° C. overnight. Cells were then treated with anti-IgM or left untreated for 6 hours. After stimulation, PBMC were fixed using CytoFix buffer and permeabilized using 0.1% Triton X-100 solution. Cells were then stained with anti-CD19 to label B (CLL) cells, anti-CD4 and anti-CD8 to label T cells, Hoechst 33342 to label nuclei, and anti-p105/p50 to label NF-kB subunit. Samples were analyzed on ImageStreamX to evaluate NF-kB nuclear localization in B cells or T cells. Frequencies of cells with nuclear enrichment of NF-kB were demonstrated in FIGS. 4 and 5. Statistical significance was determined using Student's t-test in Microsoft Excel.

It was shown that Compound A inhibited NF-κB nuclear translocation in B cells activated by anti-IgM, but not in the T cells treated with anti-IgM. However, NF-kB translocation in T cells was suppressed by Compound A in CLL blood samples treated with anti-CD3/anti-CD28 (data not shown).

### Example 7: CXCL 10 Expression Analysis on Whole Blood Samples from NHL and CLL Samples Treated with Compound a

Gene expression signatures were sought to demonstrate the effect of a MALT inhibitor from lysed whole blood of NHL and CLL patients. Peripheral blood was collected from three NHL and two CLL patients. The peripheral blood was allowed to stand overnight at room temperature, then the blood was treated for 24 hours with Compound A (200 μM) or DMSO control. The blood was then stimulated with monoclonal antibodies against CD3 and CD28 for four hours using a method similar to that described in Example 1. Prior to stimulation and following stimulation, the treated blood was transferred into a PAXgene tube (Qiagen; Hilden, Germany) and RNA was extracted using the PAXgene RNA kit. Gene expression was measured using 100 ng of RNA extracted from the whole blood in the Pan-Cancer Immune Profiling kit (NanoString; Seattle, Wash.) according to the manufacturer's instructions.

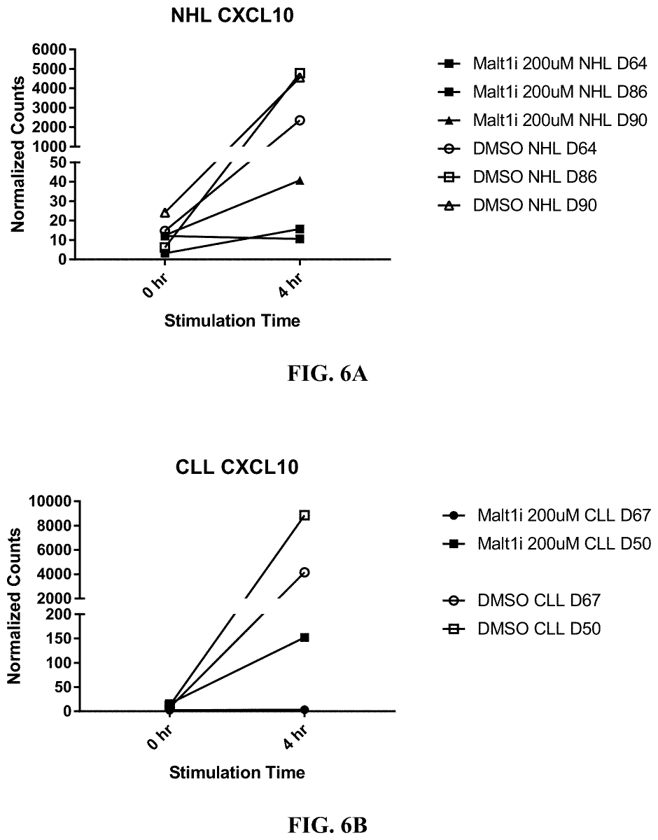

FIGS. 6A-6B show the expression levels of CXCL10, an NF-1B regulated gene, in the NHL (FIG. 6A) and CLL (FIG. 6B) samples. Stimulation of the blood samples with anti-CD3 and anti-CD28 resulted in upregulation of CXCL10 in the DMSO control samples (unfilled symbols) from all NHL and CLL patients, whereas the stimulation induced upregulation of CXCL10 was repressed in the presence of MALT1 inhibitor (filled symbols).

Expression of CXCL10 is provided as a representative gene. Tables 1-4 show lists of additional genes that can be used as an indicator of MALT1 inhibition by a MALT inhibitor. The tables comprise genes that are >2 fold up or down regulated in samples treated with MALT inhibitor relative to the DMSO controls.

TABLE 1
Genes repressed by MALT inhibitor treatment in CLL patients. Values
are log₂ fold changes between the average expression of 3 CLL
donors treated with MALT inhibitor divided by the DMSO control.

| | | log2 fold change |
|---|---|---|
| GENE SYMBOL | Gene Name | MALTi/DMSO |
| CXCL10 | C-X-C motif chemokine ligand 10(CXCL10) | −6.23761 |
| FN1 | fibronectin 1(FN1) | −3.4889 |
| CXCL9 | C-X-C motif chemokine ligand 9(CXCL9) | −3.47723 |
| CXCL11 | C-X-C motif chemokine ligand 11(CXCL11) | −2.80918 |
| IL2 | interleukin 2(IL2) | −2.63871 |
| CCL8 | C-C motif chemokine ligand 8(CCL8) | −2.36569 |
| CMKLR1 | chemerin chemokine-like receptor 1(CMKLR1) | −2.28358 |
| MSR1 | macrophage scavenger receptor 1(MSR1) | −2.28117 |
| EGR2 | early growth response 2(EGR2) | −2.10529 |
| CCL13 | C-C motif chemokine ligand 13(CCL13) | −2.07811 |
| IL21 | interleukin 21(IL21) | −1.80691 |
| EGR1 | early growth response 1(EGR1) | −1.80596 |
| IL1RN | interleukin 1 receptor antagonist(IL1RN) | −1.79666 |
| TNFSF10 | tumor necrosis factor superfamily member | −1.7951 |
| | 10(TNFSF10) | |
| IL17B | interleukin 17B(IL17B) | −1.7948 |
| MRC1 | mannose receptor, C type 1(MRC1) | −1.71364 |
| TNFRSF4 | TNF receptor superfamily member 4(TNFRSF4) | −1.70485 |
| TNFSF13B | tumor necrosis factor superfamily member | −1.70337 |
| | 13b(TNFSF13B) | |
| APOE | apolipoprotein E(APOE) | −1.64456 |
| TNFRSF13B | TNF receptor superfamily member | −1.6006 |
| | 13B(TNFRSF13B) | |
| IFNG | interferon gamma(IFNG) | −1.5641 |
| C1QA | complement C1q A chain(C1QA) | −1.55822 |
| CD36 | CD36 molecule(CD36) | −1.52077 |
| CD244 | CD244 molecule(CD244) | −1.51962 |
| CXCL6 | C-X-C motif chemokine ligand 6(CXCL6) | −1.44853 |
| CD163 | CD163 molecule(CD163) | −1.44637 |
| CCL28 | C-C motif chemokine ligand 28(CCL28) | −1.43101 |
| ULBP2 | UL16 binding protein 2(ULBP2) | −1.42264 |
| HAMP | hepcidin antimicrobial peptide(HAMP) | −1.42213 |
| TNF | tumor necrosis factor(TNF) | −1.41737 |
| IFIT2 | interferon induced protein with tetratricopeptide | −1.391 |
| | repeats 2(IFIT2) | |
| PPARG | peroxisome proliferator activated receptor | −1.37583 |
| | gamma(PPARG) | |
| OAS3 | 2′-5′-oligoadenylate synthetase 3(OAS3) | −1.35311 |
| CCL27 | C-C motif chemokine ligand 27(CCL27) | −1.34408 |
| BIRC5 | baculoviral IAP repeat containing 5(BIRC5) | −1.33518 |
| C9 | complement C9(C9) | −1.33089 |
| AXL | AXL receptor tyrosine kinase(AXL) | −1.32852 |
| MASP1 | mannan binding lectin serine peptidase 1(MASP1) | −1.31123 |
| MUC1 | mucin 1, cell surface associated(MUC1) | −1.25111 |
| CD274 | CD274 molecule(CD274) | −1.24173 |
| TLR8 | toll like receptor 8(TLR8) | −1.20602 |
| CT45A1 | cancer/testis antigen family 45 member | −1.1704 |
| | A1(CT45A1) | |
| NLRP3 | NLR family pyrin domain containing 3(NLRP3) | −1.15735 |
| CFD | complement factor D(CFD) | −1.14569 |
| NOS2 | Nitric Oxide Synthase 2 (NOS2A) | −1.08816 |
| CCR3 | C-C motif chemokine receptor 3(CCR3) | −1.08522 |
| CD70 | CD70 molecule(CD70) | −1.06972 |
| BST2 | bone marrow stromal cell antigen 2(BST2) | −1.06542 |
| RELA | RELA proto-oncogene, NF-kB subunit(RELA) | −1.06425 |
| TPTE | transmembrane phosphatase with tensin | −1.04304 |
| | homology(TPTE) | |
| IFI35 | interferon induced protein 35(IFI35) | −1.02516 |
| IL7 | interleukin 7(IL7) | −1.02351 |
| MAGEA1 | MAGE family member A1(MAGEA1) | −1.00155 |

TABLE 2
Genes upregulated by MALT inhibitor treatment in CLL patients. Values
are log₂ fold changes between the average expression of 3 CLL donors
treated with MALT inhibitor divided by the DMSO control

| | | log2 fold change |
|---|---|---|
| GENE SYMBOL | Gene Name | MALTi/DMSO |
| PLAU | plasminogen activator, urokinase(PLAU) | 3.725443 |
| IL6 | interleukin 6(IL6) | 2.489794 |
| CXCL5 | C-X-C motif chemokine ligand 5(CXCL5) | 2.342973 |
| C3 | complement C3(C3) | 2.212404 |
| CXCL3 | C-X-C motif chemokine ligand 3(CXCL3) | 2.199427 |
| CXCL8 | chemokine (C-X-C motif) ligand 8 (IL8) | 2.116282 |
| DUSP4 | dual specificity phosphatase 4(DUSP4) | 2.032355 |
| VEGFA | vascular endothelial growth factor A(VEGFA) | 1.960556 |
| IFNB1 | interferon beta 1(IFNB1) | 1.934917 |
| KIR3DS1 | KIR_Inhibiting_Subgroup_1 | 1.841838 |
| PLAUR | plasminogen activator, urokinase receptor(PLAUR) | 1.811316 |
| TREM1 | triggering receptor expressed on myeloid cells | 1.76384 |
| | 1(TREM1) | |
| IL1R2 | interleukin 1 receptor type 2(IL1R2) | 1.691364 |
| SERPINB2 | serpin family B member 2(SERPINB2) | 1.651362 |
| CXCL2 | C-X-C motif chemokine ligand 2(CXCL2) | 1.61791 |
| IRAK2 | interleukin 1 receptor associated kinase 2(IRAK2) | 1.594393 |
| C3AR1 | complement C3a receptor 1(C3AR1) | 1.566417 |
| KIR3DL1 | killer cell immunoglobulin like receptor, three Ig | 1.487092 |
| | domains and long cytoplasmic tail 1(KIR3DL1) | |
| SLC11A1 | solute carrier family 11 member 1(SLC11A1) | 1.47976 |
| CCRL2 | C-C motif chemokine receptor like 2(CCRL2) | 1.432187 |
| JAML | junction adhesion molecule like (AMICA) | 1.421695 |
| CCL23 | C-C motif chemokine ligand 23(CCL23) | 1.388691 |
| IL1RAP | interleukin 1 receptor accessory protein(IL1RAP) | 1.373874 |
| CCR4 | C-C motif chemokine receptor 4(CCR4) | 1.362528 |
| TNFSF18 | tumor necrosis factor superfamily member | 1.33906 |
| | 18(TNFSF18) | |
| ATM | ATM serine/threonine kinase(ATM) | 1.337823 |
| MAGEA4 | MAGE family member A4(MAGEA4) | 1.337288 |
| THBS1 | thrombospondin 1(THBS1) | 1.326325 |
| LGALS3 | galectin 3(LGALS3) | 1.297857 |
| CCL3L1 | C-C motif chemokine ligand 3 like 1(CCL3L1) | 1.216666 |
| CEBPB | CCAAT/enhancer binding protein beta(CEBPB) | 1.208096 |
| IL1RL2 | interleukin 1 receptor like 2(IL1RL2) | 1.194175 |
| THBD | thrombomodulin(THBD) | 1.179067 |
| IL24 | interleukin 24(IL24) | 1.173913 |
| ADORA2A | adenosine A2a receptor(ADORA2A) | 1.172815 |
| CXCR4 | C-X-C motif chemokine receptor 4(CXCR4) | 1.16789 |
| LY9 | lymphocyte antigen 9(LY9) | 1.166111 |
| PPBP | pro-platelet basic protein(PPBP) | 1.151512 |
| CCL16 | C-C motif chemokine ligand 16(CCL16) | 1.113953 |
| IL15RA | interleukin 15 receptor subunit alpha(IL15RA) | 1.078907 |
| IFNA8 | interferon alpha 8(IFNA8) | 1.071804 |
| FCER1G | Fc fragment of IgE receptor Ig(FCER1G) | 1.068402 |
| IFNGR1 | interferon gamma receptor 1(IFNGR1) | 1.058767 |
| S100A8 | S100 calcium binding protein A8(S100A8) | 1.028829 |
| CD209 | CD209 molecule(CD209) | 1.026092 |
| TNFRSF14 | TNF receptor superfamily member 14(TNFRSF14) | 1.018935 |
| OSM | oncostatin M(OSM) | 1.006489 |

TABLE 3
Genes repressed by MALT inhibitor treatment in NHL patients. Values
are log₂ fold changes between the average expression of 3 NHL
donors treated with MALT inhibitor divided by the DMSO control.

| | | log2 fold change |
|---|---|---|
| GENE SYMBOL | Gene Name | MALTi/DMSO |
| CXCL10 | C-X-C motif chemokine ligand 10(CXCL10) | −7.44425 |
| FN1 | fibronectin 1(FN1) | −6.39939 |
| CXCL9 | C-X-C motif chemokine ligand 9(CXCL9) | −4.51545 |
| CCL8 | C-C motif chemokine ligand 8(CCL8) | −4.34886 |
| MSR1 | macrophage scavenger receptor 1(MSR1) | −3.86464 |
| CXCL6 | C-X-C motif chemokine ligand 6(CXCL6) | −3.66149 |
| IL2 | interleukin 2(IL2) | −2.86532 |
| XCL2 | X-C motif chemokine ligand 2(XCL2) | −2.38716 |
| SPP1 | secreted phosphoprotein 1(SPP1) | −2.38647 |
| CD163 | CD163 molecule(CD163) | −2.36691 |
| FCGR1A | Fc fragment of IgG receptor Ia(FCGR1A) | −2.33665 |
| SERPING1 | serpin family G member 1(SERPING1) | −2.1882 |
| APOE | apolipoprotein E(APOE) | −2.14008 |
| TNF | tumor necrosis factor(TNF) | −2.04196 |
| IL21 | interleukin 21(IL21) | −1.98795 |
| EGR2 | early growth response 2(EGR2) | −1.8383 |
| TNFRSF11A | TNF receptor superfamily member | −1.82103 |
| | 11a(TNFRSF11A) | |
| DUSP6 | dual specificity phosphatase 6(DUSP6) | −1.79174 |
| NLRP3 | NLR family pyrin domain containing 3(NLRP3) | −1.76735 |
| TNFSF10 | tumor necrosis factor superfamily member | −1.71238 |
| | 10(TNFSF10) | |
| TICAM2 | toll like receptor adaptor molecule 2(TICAM2) | −1.70777 |
| IRF8 | interferon regulatory factor 8(IRF8) | −1.65661 |
| TNFRSF9 | TNF receptor superfamily member 9(TNFRSF9) | −1.64241 |
| CXCL11 | C-X-C motif chemokine ligand 11(CXCL11) | −1.63743 |
| PDCD1LG2 | programmed cell death 1 ligand 2(PDCD1LG2) | −1.63696 |
| CD36 | CD36 molecule(CD36) | −1.59397 |
| HLA-DMB | major histocompatibility complex, class II, DM | −1.5632 |
| | beta(HLA-DMB) | |
| CD86 | CD86 molecule(CD86) | −1.56006 |
| FCGR2B | Fc fragment of IgG receptor IIb(FCGR2B) | −1.54176 |
| IRF1 | interferon regulatory factor 1(IRF1) | −1.53314 |
| CMKLR1 | chemerin chemokine-like receptor 1(CMKLR1) | −1.50087 |
| CASP10 | caspase 10(CASP10) | −1.48884 |
| CD274 | CD274 molecule(CD274) | −1.45834 |
| CFD | complement factor D(CFD) | −1.45802 |
| CAMP | cathelicidin antimicrobial peptide(CAMP) | −1.44664 |
| FCER1A | Fc fragment of IgE receptor la(FCER1A) | −1.42921 |
| IFNL2 | interferon lambda 2(IFNL2) | −1.42398 |
| TNFSF8 | tumor necrosis factor superfamily member | −1.41322 |
| | 8(TNFSF8) | |
| MBL2 | mannose binding lectin 2(MBL2) | −1.38957 |
| CD160 | CD160 molecule(CD160) | −1.36681 |
| TNFRSF4 | TNF receptor superfamily member 4(TNFRSF4) | −1.33342 |
| MEF2C | myocyte enhancer factor 2C(MEF2C) | −1.33179 |
| CCL7 | C-C motif chemokine ligand 7(CCL7) | −1.28969 |
| CCR2 | C-C motif chemokine receptor 2(CCR2) | −1.27647 |
| TAP1 | transporter 1, ATP binding cassette subfamily B | −1.24684 |
| | member(TAP1) | |
| HLA-DMA | major histocompatibility complex, class II, DM | −1.22772 |
| | alpha(HLA-DMA) | |
| MS4A1 | membrane spanning 4-domains A1(MS4A1) | −1.21431 |
| STAT1 | signal transducer and activator of transcription | −1.19315 |
| | 1(STAT1) | |
| A2M | alpha-2-macroglobulin(A2M) | −1.17568 |
| CCL2 | C-C motif chemokine ligand 2(CCL2) | −1.14876 |
| MAGEA3 | MAGE family member A3(MAGEA3) | −1.14205 |
| C2 | complement C2(C2) | −1.11037 |
| TLR8 | toll like receptor 8(TLR8) | −1.09767 |
| FCGR3A | Fc fragment of IgG receptor IIIa(FCGR3A) | −1.09554 |
| PASD1 | PAS domain containing 1(PASD1) | −1.05537 |
| ALCAM | activated leukocyte cell adhesion | −1.05433 |
| | molecule(ALCAM) | |
| CXCL1 | C-X-C motif chemokine ligand 1(CXCL1) | −1.03739 |
| NUBP1 | nucleotide binding protein 1(NUBP1) | −1.03215 |
| CX3CR1 | C-X3-C motif chemokine receptor 1(CX3CR1) | −1.02947 |
| SPANXB1 | SPANX family member B1(SPANXB1) | −1.02926 |
| CD1D | CD1d molecule(CD1D) | −1.00708 |
| LTB | lymphotoxin beta(LTB) | −1.00365 |

TABLE 4
Genes upregulated by MALT inhibitor treatment in NHL patients. Values
are log₂ fold changes between the average expression of 3 NHL donors
treated with MALT inhibitor divided by the DMSO control.

| | | log2 fold change |
|---|---|---|
| GENE SYMBOL | Gene Name | malt/DMSO |
| IL6 | interleukin 6(IL6) | 4.424478 |
| PLAU | plasminogen activator, urokinase(PLAU) | 4.346512 |
| IL24 | interleukin 24(IL24) | 4.090957 |
| CD22 | CD22 molecule(CD22) | 3.839703 |
| CXCL8 | chemokine (C-X-C motif) ligand 8 | 3.002058 |
| PTGS2 | prostaglandin-endoperoxide synthase 2(PTGS2) | 2.808481 |
| CXCR4 | C-X-C motif chemokine receptor 4(CXCR4) | 2.599085 |
| IL10 | interleukin 10(IL10) | 2.584502 |
| PLAUR | plasminogen activator, urokinase | 2.258011 |
| | receptor(PLAUR) | |
| VEGFA | vascular endothelial growth factor A(VEGFA) | 2.250191 |
| IL12B | interleukin 12B(IL12B) | 2.207348 |
| OSM | oncostatin M(OSM) | 2.199139 |
| SLC11A1 | solute carrier family 11 member 1(SLC11A1) | 2.137152 |
| EBI3 | Epstein-Barr virus induced 3(EBI3) | 2.05429 |
| IL3RA | interleukin 3 receptor subunit alpha(IL3RA) | 1.998028 |
| ADA | adenosine deaminase(ADA) | 1.912374 |
| IRAK2 | interleukin 1 receptor associated kinase | 1.902267 |
| | 2(IRAK2) | |
| CCL23 | C-C motif chemokine ligand 23(CCL23) | 1.874754 |
| BAGE | B melanoma antigen(BAGE) | 1.850765 |
| IL19 | interleukin 19(IL19) | 1.850424 |
| CXCL2 | C-X-C motif chemokine ligand 2(CXCL2) | 1.825243 |
| LGALS3 | galectin 3(LGALS3) | 1.760822 |
| TNFRSF8 | TNF receptor superfamily member 8(TNFRSF8) | 1.709077 |
| CCL3 | C-C motif chemokine ligand 3(CCL3) | 1.66063 |
| C3 | complement C3(C3) | 1.647978 |
| CCL3L1 | C-C motif chemokine ligand 3 like 1(CCL3L1) | 1.633286 |
| CCL19 | C-C motif chemokine ligand 19(CCL19) | 1.557437 |
| IFNGR1 | interferon gamma receptor 1(IFNGR1) | 1.550933 |
| LIF | leukemia inhibitory factor(LIF) | 1.527946 |
| IFIT1 | interferon induced protein with | 1.524019 |
| | tetratricopeptide repeats 1(IFIT1) | |
| CCL24 | C-C motif chemokine ligand 24(CCL24) | 1.48059 |
| ITGB3 | integrin subunit beta 3(ITGB3) | 1.447969 |
| IL23A | interleukin 23 subunit alpha(IL23A) | 1.439153 |
| CD83 | CD83 molecule(CD83) | 1.411104 |
| BCL6 | B-cell CLL/lymphoma 6(BCL6) | 1.397515 |
| CSF1 | colony stimulating factor 1(CSF1) | 1.396813 |
| FCER1G | Fc fragment of IgE receptor Ig(FCER1G) | 1.386864 |
| THBD | thrombomodulin(THBD) | 1.369203 |
| VEGFC | vascular endothelial growth factor C(VEGFC) | 1.346309 |
| TFRC | transferrin receptor(TFRC) | 1.34324 |
| SLAMF7 | SLAM family member 7(SLAMF7) | 1.333665 |
| IL2RA | interleukin 2 receptor subunit alpha(IL2RA) | 1.317348 |
| BCL2L1 | BCL2 like 1(BCL2L1) | 1.253259 |
| SELE | selectin E(SELE) | 1.234141 |
| S100B | S100 calcium binding protein B(S100B) | 1.233235 |
| RORA | RAR related orphan receptor A(RORA) | 1.21919 |
| TNFRSF1B | TNF receptor superfamily member | 1.218378 |
| | 1B(TNFRSF1B) | |
| TNFSF15 | tumor necrosis factor superfamily member | 1.216164 |
| | 15(TNFSF15) | |
| CCRL2 | C-C motif chemokine receptor like 2(CCRL2) | 1.201969 |
| ATM | ATM serine/threonine kinase(ATM) | 1.197985 |
| RUNX3 | runt related transcription factor 3(RUNX3) | 1.176228 |
| IFI27 | interferon alpha inducible protein 27(IFI27) | 1.171608 |
| PRG2 | proteoglycan 2, pro eosinophil major basic | 1.167997 |
| | protein(PRG2) | |
| CEBPB | CCAAT/enhancer binding protein beta(CEBPB) | 1.163182 |
| ITGA1 | integrin subunit alpha 1(ITGA1) | 1.156243 |
| CDH1 | cadherin 1(CDH1) | 1.137557 |
| CCL22 | C-C motif chemokine ligand 22(CCL22) | 1.105017 |
| LYN | LYN proto-oncogene, Src family tyrosine | 1.103615 |
| | kinase(LYN) | |
| NOS2 | Nitric Oxide Synthase 2 (NOS2A) | 1.096731 |
| IFNB1 | interferon beta 1(IFNB1) | 1.087681 |
| KLRB1 | killer cell lectin like receptor B1(KLRB1) | 1.085404 |
| IL1R1 | interleukin 1 receptor type 1(IL1R1) | 1.059275 |
| FOS | Fos proto-oncogene, AP-1 transcription factor | 1.046243 |
| | subunit(FOS) | |
| SELPLG | selectin P ligand(SELPLG) | 1.029262 |
| CSF3 | colony stimulating factor 3(CSF3) | 1.020689 |
| CCL13 | C-C motif chemokine ligand 13(CCL13) | 1.004443 |

### Example 8: IL2 Expression Analysis on Purified T Cells from NHL Samples Treated with Compound A

The effect of a MALT inhibitor was analyzed utilizing gene expression signatures from purified T-cells and PBMCs from Non-Hodgkin's lymphoma patients. Peripheral blood was collected from five NHL patients, and the peripheral blood was allowed to stand overnight. Then, the peripheral blood was treated for 24 hours with a MALT inhibitor or DMSO control. The blood was then stimulated with monoclonal antibodies against CD3 and CD28 for six hours. Prior to stimulation and following stimulation, PBMC were purified from the treated blood using a ficoll density gradient and T-cells were purified using CD3 Beads (Miltenyi) using the manufacturers protocol. Purified cells were lysed in RLTplus (Qiagen) and RNA was extracted from the purified cells using an AllPrepkit (Qiagen). Gene expression was measured using 100 ng of RNA in the Pan-Cancer Immune Profiling kit (NanoString) according to the manufacturer's instructions.

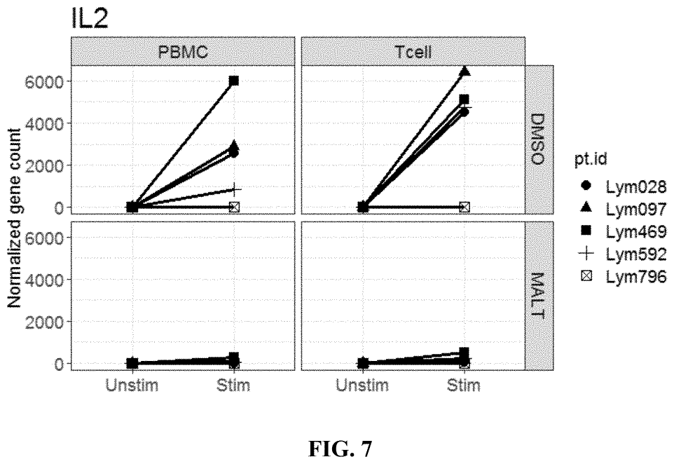

FIG. 7 shows the expression levels of IL2, an NF-κB regulated gene, in the T-cell and PBMC fractions of the blood prior to and following stimulation. In the DMSO controls (FIG. 7, upper panels) stimulation of blood results in the upregulation of IL2 in both T-cells and PBMC for most donors, whereas stimulation induced upregulation of IL2 is repressed in the presence of MALT inhibitor (FIG. 7, lower panels). Analysis of IL2 expression in purified T cells demonstrated more uniform induction of IL2 expression in the DMSO control samples from all patient samples tested. This indicates that for certain marker genes, such as IL2, PBMCs can be further separated (e.g., T cells can be purified) following stimulation and MALT inhibitor treatment to provide more reproducible results on the gene expression analysis.

IL2 gene is provided as a representative gene. Expression of other marker genes can be analyzed in similar manner with T cells purified from the blood samples of CLL or NHL patients.

### Example 9: Gene Expression Analysis of PBMCs Purified from NHL Patients Treated with Compound a without Stimulation of the PBMCs

The effect of a MALT inhibitor was demonstrated by analyzing gene expression signatures from PBMCs purified from NHL patients without stimulation of the blood cells. Peripheral blood was collected from five NHL patients, and the peripheral blood was allowed to stand overnight. Then, the peripheral blood was treated for 24 hours with a MALT inhibitor or DMSO control. PBMCs were purified from the unstimulated blood using a ficoll density gradient and T-cells were purified using CD3 Beads (Miltenyi) using the manufacturers protocol. Purified cells were lysed in RLTplus (Qiagen) and RNA was extracted from the purified cells using an AllPrep kit (Qiagen). Gene expression was measured using 100 ng of RNA in the Pan-Cancer Immune Profiling kit (NanoString) according to the manufacturer's instructions. Gene expression signatures were compared between the MALT inhibitor treated and DMSO treated samples.

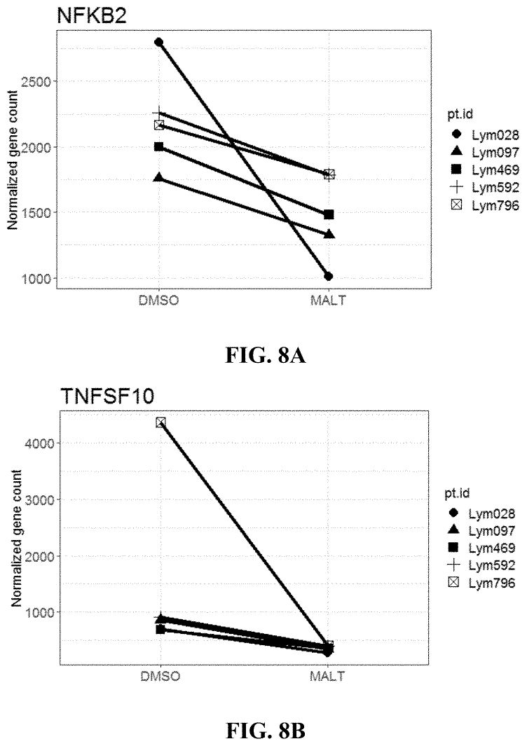

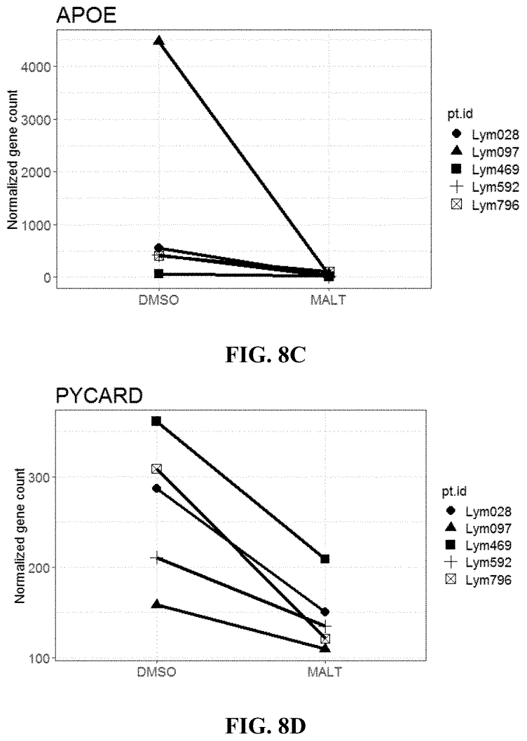

FIGS. 8A-8D show genes (e.g., NF-κB2 (FIG. 8A), TNFSF10 (FIG. 8B), APOE (FIG. 8C), and PYCARD (FIG. 8D)) repressed by MALT inhibition in the absence of cell stimulation in purified T-cells (FIGS. 8A and 8B) and in purified PBMCs (FIGS. 8C and 8D). These results indicate that the efficacy of a MALT1 inhibitor can be assessed by gene expression analysis on certain marker genes, such as NF-κB2, TNFSF10, APOE, and PYCARD, from T cells or PBMCs purified from the blood of the patients without additional stimulation of the T cells or PBMCs in vitro. However, large patient to patient variability was observed.

Genes shown in FIGS. 8A-8D are provided as representatives. Other marker genes can also be analyzed in similar manner with T cells or PBMCs purified from the blood samples of CLL or NHL patients.

### Example 10: NF-kB Translocation in T Cells from Peripheral Blood of NHL Patients Upon Ex Vivo Stimulation with Different Agents

Whole blood samples of ten B-cell NHL patients were tested when the blood sample was subjected to stimulation with different agents, e.g., CD3/CD28 or PMA/ionomycin, or PBS as a control. Briefly, NHL donor blood was collected in 10 mL NaHep tubes and transported in refrigerated state (cold packs). Upon arrival at the lab, aliquots of the blood were diluted with RPMI 1640+10% FBS medium and treated with Compound A (100 μM) or DMSO (control) for 2 hours. Blood was then stimulated either with anti-CD3 ((UCHT1 clone; BioLegend, cat. #300465; San Diego, Calif.) and anti-CD28 antibodies (ANC28.1 clone; Ancell, cat. #177-024; British Columbia, Canada) antibodies at a final concentration of 1 μg/mL each or PMA (20 ng/mL) and Ionomycin (1 μg/mL) or left unstimulated (PBS only control) for 4 hours. Following stimulation, the blood was lysed with RBC Lysis buffer and the leukocytes were fixed in 4.2% paraformaldehyde (PFA). Upon fixation, cells were permeabilized with 0.1% Triton X-100 and stained for CD3, NF-kB (p65 and p50/p105) with the respective antibodies and stained the nuclei with Hoechst 33342. Samples were collected on ImageStream MkII (Luminex) and the images were analyzed in IDEAS software (Luminex).

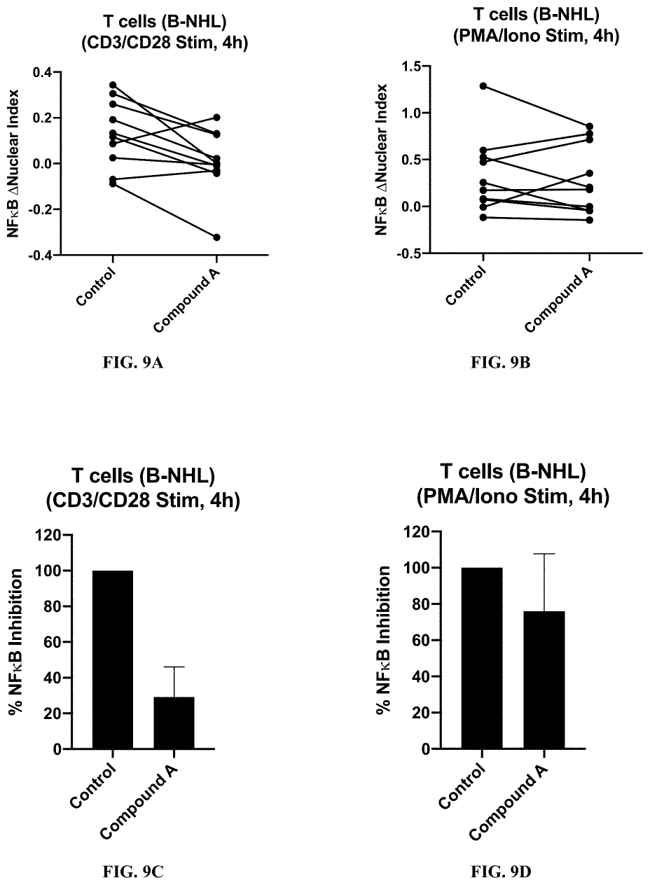

The delta nuclear index in T cells was obtained for NF-kB nuclear translocation by calculating the difference between the median value of nuclear index in CD3+ T cells from the unstimulated (control) and the stimulated (CD3/CD28 stim or PMA/Iono stim) conditions, and the obtained values were corrected for baseline levels in unstimulated samples (FIGS. 9A and 9B). The mean values of delta nuclear index were normalized to control (DMSO treatment) and represented as percentage of inhibition (FIGS. 9C and 9D). Relative percentage of inhibition was obtained by normalizing the delta nuclear index values for NF-kB translocation in the cells treated with Compound A to the delta nuclear index values for NF-kB translocation in the cells treated with DMSO. Data in FIGS. 9C and 9D are mean with standard error of means.

Results of this study showed that a MALT1 inhibitor (Compound A) inhibited NF-kB nuclear translocation in T cells of NHL patients activated by anti-CD3 and anti-CD28 antibodies or PMA and Ionomycin.

### Example 11: Gene Expression Signatures of MALTi Activity when Peripheral Blood is Treated with Lymphocyte-Stimulating Agents

It is possible that stimulation of T-cells with different agents can produce different optimal gene expression signatures to demonstrate the activity of a MALT1 inhibitor. To obtain a robust gene signature of MALT1 inhibitor activity, whole blood of nine NHL patients which was stimulated with CD3/CD28, PMA/ionomycin, or PBS as a control was tested. Briefly, NHL donor blood was collected in 10 mL sodium heparin tubes and transported in refrigerated state (cold packs). Upon arrival at the lab, aliquots of the blood were diluted with RPMI+10% FBS medium and treated with 100 μM Compound A or DMSO (control) for 2 hours. Then, aliquots were stimulated with CD3 and CD28 antibodies (1 ug/ml each), PMA (20 ng/ml) and Ionomycin (1 μg/mL), or unstimulated (PBS) for four hours. Following stimulation, the treated blood was transferred into a PAXgene tube (Qiagen; Hilden, Germany) and RNA was extracted using the PAXgene RNA kit.

Gene expression was measured using 100 ng of RNA extracted from the whole blood in the Pan-Cancer Immune Profiling kit (NanoString; Seattle, Wash.) according to the manufacturer's instructions. Gene expression was normalized across samples using NanoString nSolver software. Analyses of the Nanostring gene expression data were performed in the R statistical environment, using the ‘limma’ package, to determine whether certain genes are significantly different in their expression levels in the presence of Compound A following either stimulation condition. Briefly, log 2-scaled, normalized NanoString counts were fit to a linear model (which incorporated the patient of origin) and moderated t-statistics, along with the associated Benjamini-Hochberg (BH) corrected p-values, were computed by empirical Bayes moderation of the standard errors towards a common value.

The following genes had a fold-change of <1.5 and an adjusted p-value <0.05 when samples were treated with Compound A after CD3/CD28 stimulation: IL2, TNFRSF18, CD40LG, ICOS, CCL4, CTLA4, CCL20, CCL1, TNFRSF4, CCL3L1, IL6, CCL3, TNF, IL4, FEZ1, LTA, IL9, IFNG, L3, IL1A, CCL8, CD163, CSF2, MRC1, IL22, and IL13, while the following genes had a fold-change >1.5 and an adjusted p-value <0.05: IL19, THBS1, ADA, & PECAM1.

In the PMA-stimulated samples, the following genes were downregulated at the previously specified cutoff levels when samples were treated with Compound A: ICOS, POU2F2, CCR4, and CTLA4, while no genes were significantly upregulated. Analyses of samples treated with only PBS showed that (a) the following genes had a fold-change <1.5 and an adjusted p-value <0.05: SPP1 and FN1, while the following genes had a fold-change >1.5 and an adjusted p-value <0.05: THBS1, SERPINB2, MME, and IL10.

Based on these results, a list of genes differentially expressed in patient samples treated with MALT1 inhibitor and then exposed to lymphocyte-stimulating agents was compiled by combining genes associated with the PMA/ionomycin and CD3/CD28 experiments, then subtracting any genes associated with the PBS-only experiment. Therefore, classification- and/or regression-based approaches can be used to determine the degree of MALT1 inhibitor activity when peripheral blood is treated with lymphocyte-stimulating agents based upon the expression levels of one or more of the following genes: IL2, TNFRSF18, CD40LG, ICOS, CCL4, CTLA4, CCL20, CCL1, TNFRSF4, CCL3L1, IL6, CCL3, TNF, IL4, FEZ1, LTA, IL9, IFNG, IL3, IL1A, CCL8, CD163, CSF2, MRC, IL22, IL13, POU2F2, CCR4, IL19, ADA, and PECAM1.

### Example 12: Clinical Study

A first in human (FIH), open-label, multicenter, Phase 1 study is conducted to evaluate the safety, PK, PD, and preliminary clinical activity of Compound A monotherapy administered to adult participants with advanced B-lymphocytic malignancies who previously received or are ineligible for standard treatment options. Compound A will be administered orally once daily on an outpatient basis. Throughout treatment administration, routine study procedures and laboratory assessments will be performed to monitor safety as well as to evaluate clinical activity, PK, and PD endpoints.

Biomarker samples will be collected to evaluate the Pharmacodynamic (PD) of Compound A. Samples collected for biomarker evaluations include, for example, serial blood samples. Samples can be evaluated for PD markers to determine the effect of MALT1 inhibition by Compound A. Flow cytometry-based evaluations of immune cells subsets from the blood will also be performed to determine exploratory biomarkers. Whole blood will be collected on Cycle 1 Day 1 predose for baseline assessment.

The whole blood sample be used for DNA sequencing using a targeted gene panel and whole exome sequencing as needed. Retrospective analysis to correlate mutational status to clinical response will be performed to identify predictive biomarkers of clinical response and potential mechanisms of resistance, including TNFAIP3/A20 deletion or mutation. All samples from the DLBCL cohort will be sent to a central laboratory for testing using next-generation sequencing (NGS) analysis for mutations in CD79b and CARD11. The results of the central laboratory will be considered final in the event there is a discrepancy between the results of local testing and the central laboratory.

Blood intended for ex vivo testing is collected from B-NHL subjects undergoing MALT1 inhibitor treatment into a 10 mL sodium heparin tube and transported to a clinical research organization at ambient temperature for next day delivery. Upon receipt, the blood sample is aliquoted evenly into two 15 mL conical tubes (Corning, cat. #430052) and mixed with equal volumes of room-temperature RPMI 1640 medium with 25 mM HEPES (Life Technologies, cat. #72400-047), supplemented with 10% heat-inactivated fetal bovine serum (Life Technologies, cat. #16140-071). One tube is labeled “Stimulated” and another labeled “Control.”

An anti-CD3 antibody (UCHT1 clone) and an anti-CD28 antibody (ANC28.1 clone) are added to the “Stimulated” tube at a final concentration of 1 μg/mL each. An equivalent volume of vehicle control (dPBS, Life Technologies, cat. #14190144) is added to the “Control” tube. The tubes are capped tightly and mixed well by inverting a few times. The tubes are incubated for 6 hours in a humidified incubator at 37° C. with 5% CO₂ with gentle mixing on a rocker.

After the incubation, 2.5 mL of the “Stimulated” and the “Control” blood mixtures are transferred into labeled PAXgene tubes (BD Biosciences, cat. #762165; Plymouth Meeting, PA) and 1 mL of the “Stimulated” and the “Control” blood mixtures are transferred into labeled Smart Tubes (Fisher Scientific, cat. #501351690; Waltham, Mass.), yielding two tubes for each condition. Fixative is mixed well in the Smart Tubes by inverting three times. The PAXgene tubes and the Smart Tubes are incubated on the benchtop for 10 minutes at room temperature. Then the tubes are transferred promptly into the −80° C. freezer. The samples are maintained at −80° C. or on dry ice at all times until sample analysis.

Blood intended for ex vivo testing is collected from CLL subjects undergoing MALT1 inhibitor treatment into a 10 mL sodium heparin tube and transported to a clinical research organization at ambient temperature for next day delivery. Upon receipt, the blood sample is aliquoted evenly into two 15 mL conical tubes and mixed with equal volumes of room-temperature RPMI 1640 medium with 25 mM HEPES, supplemented with 10% heat-inactivated fetal bovine serum. One tube is labeled “Stimulated” and another labeled “Control.” An anti-Human IgM (F(ab′)2 fragment, Jackson ImmunoResearch, cat. #109-006-129) is added to the “Stimulated” tube at the final concentration of 15 μg/mL. An equivalent volume of vehicle control (dPBS) is added to the “Control” tube. The tubes are capped tightly and mixed well by inverting a few times. The tubes are incubated for 30 minutes in a humidified incubator at 37° C. with 5% CO₂ with gentle mixing on a rocker.

After the incubation, 2.5 mL of the “Stimulated” and the “Control” blood mixtures are transferred into labeled PAXgene tubes and 1 mL of the “Stimulated” and the “Control” blood mixtures are transferred into labeled Smart Tubes, yielding two tubes for each condition. Fixative is mixed well in the Smart Tubes by inverting three times. The PAXgene tubes and the Smart Tubes are incubated on the benchtop for 10 minutes at room temperature. Then the tubes are transferred promptly into the −80° C. freezer. The samples are maintained at −80° C. or on dry ice at all times until sample analysis.

CLL blood samples can also be stimulated with anti-human CD3 and anti-human CD28 to induce activation of peripheral T cells, using a method similar to that described above for the B-NHL blood samples.

The samples containing activated peripheral T cells or circulating B-CLL cells will be used in NF-κB nuclear translocation assays and/or marker gene expression assays according to methods described herein.

### EMBODIMENTS

1. A method of predicting a response to a MALT1 inhibitor in a subject in need thereof comprising:

(a) measuring a changed level of NF-kB nuclear translocation in a subject's test sample that has been previously exposed to the MALT1 inhibitor;

(b) measuring a changed level of NF-kB nuclear translocation in a subject's control sample that has not been previously exposed to the MALT1 inhibitor; and

(c) comparing the changed level of NF-kB nuclear translocation in (a) to (b), wherein a decrease in the changed level of NF-kB nuclear translocation in (a) is predictive of a positive response to the MALT1 inhibitor in the subject.

1a. A MALT1 inhibitor for use in a method of treating and/or diagnosing in vivo a MALT1-mediated disease in a subject, wherein the subject is predicted to be responsive to the MALT1 inhibitor by the method comprising:

(a) measuring a changed level of NF-kB nuclear translocation in a subject's test sample that has been previously exposed to the MALT1 inhibitor;

(b) measuring a changed level of NF-kB nuclear translocation in a subject's control sample that has not been previously exposed to the MALT1 inhibitor; and

(c) comparing the changed level of NF-kB nuclear translocation in (a) to (b), wherein a decrease in the changed level of NF-kB nuclear translocation in (a) is predictive of a positive response to the MALT1 inhibitor in the subject.

2. A method of monitoring an efficacy of an ongoing MALT1 inhibitor therapy in a subject in need thereof comprising:

(a) measuring a changed level of NF-kB nuclear translocation in a subject's test sample that has been previously exposed to a MALT1 inhibitor;

(b) measuring a changed level of NF-kB nuclear translocation in a subject's control sample that has not been previously exposed to a MALT1 inhibitor; and

(c) comparing the changed level of NF-kB nuclear translocation in (a) to (b), wherein a decrease in the changed level of NF-kB nuclear translocation in (a) is indicative of efficacy of the MALT1 inhibitor therapy in the subject.

2a. A MALT1 inhibitor for use in a method of treating and/or diagnosing in vivo a MALT1-mediated disease in a subject, wherein the subject is monitored for efficacy of an ongoing MALT1 inhibitor therapy by the method comprising:

(a) measuring a changed level of NF-kB nuclear translocation in a subject's test sample that has been previously exposed to a MALT1 inhibitor;

(b) measuring a changed level of NF-kB nuclear translocation in a subject's control sample that has not been previously exposed to a MALT1 inhibitor; and

(c) comparing the changed level of NF-kB nuclear translocation in (a) to (b), wherein a decrease in the changed level of NF-kB nuclear translocation in (a) is indicative of efficacy of the MALT1 inhibitor therapy in the subject.

3. A method of treating a cancer or a MALT1-mediated disease in a subject in need thereof comprising:

(a) measuring a changed level of NF-kB nuclear translocation in a subject's test sample that has been previously exposed to a MALT1 inhibitor;

(b) measuring a changed level of NF-kB nuclear translocation in a subject's control sample that has not been previously exposed to a MALT1 inhibitor;

(c) comparing the changed level of NF-kB nuclear translocation in (a) to (b); and

(d) administering a lower dose of MALT1 inhibitor to the subject if the changed level of NF-kB nuclear translocation in (a) is less than (b), and administering a higher dose of MALT1 inhibitor to the subject if the changed level of NF-kB nuclear translocation in (a) is not less than (b).

3a. A MALT1 inhibitor for use in a method of treating and/or diagnosing in vivo cancer or a MALT1-mediated disease in a subject comprising:

(a) measuring a changed level of NF-kB nuclear translocation in a subject's test sample that has been previously exposed to a MALT1 inhibitor;

(b) measuring a changed level of NF-kB nuclear translocation in a subject's control sample that has not been previously exposed to a MALT1 inhibitor;

(c) comparing the changed level of NF-kB nuclear translocation in (a) to (b); and the method further comprises administration of a lower dose of MALT1 inhibitor to the subject if the changed level of NF-kB nuclear translocation in (a) is less than (b), and administration of a higher dose of MALT1 inhibitor to the subject if the changed level of NF-kB nuclear translocation in (a) is not less than (b).

4. A method of designing a drug regimen to treat cancer or a MALT1-mediated disease in a subject in need thereof comprising:

(a) measuring a changed level of NF-kB nuclear translocation in a subject's test sample that has been previously exposed to a MALT1 inhibitor;

(b) measuring a changed level of NF-kB nuclear translocation in a subject's control sample that has not been previously exposed to a MALT1 inhibitor;

(c) comparing the changed level of NF-kB nuclear translocation in (a) to (b); and (d) administering a second therapeutic agent to the subject if changed level of NF-kB nuclear translocation in (a) is not less than (b).

4a. A MALT1 inhibitor for use in a method of treating and/or diagnosing in vivo a MALT1-mediated disease in a subject, wherein a drug regimen for the MALT1 inhibitor is designed by the method comprising:

(a) measuring a changed level of NF-kB nuclear translocation in a subject's test sample that has been previously exposed to a MALT1 inhibitor;

(b) measuring a changed level of NF-kB nuclear translocation in a subject's control sample that has not been previously exposed to a MALT1 inhibitor;

(c) comparing the changed level of NF-kB nuclear translocation in (a) to (b); and the method further comprises administration of a second therapeutic agent to the subject if changed level of NF-kB nuclear translocation in (a) is not les than (b).

5. A method of modifying the dose and/or frequency of dosing of a MALT1 inhibitor in a subject suffering from cancer or a MALT1-mediated disease comprising:

(a) measuring a changed level of NF-kB nuclear translocation in a subject's test sample that has been previously exposed to the MALT1 inhibitor;

(b) measuring a changed level of NF-kB nuclear translocation in a subject's control sample that has not been previously exposed to the MALT1 inhibitor;

(c) comparing the changed level of NF-kB nuclear translocation in (a) to (b); and

(d) reducing a dosing frequency of the MALT1 inhibitor if the changed level of NF-kB nuclear translocation in (a) is less than (b), and increasing the dosing frequency of the MALT1 inhibitor if the changed level of NF-kB nuclear translocation in (a) is not less than (b).

5a. A MALT1 inhibitor for use in a method of treating and/or diagnosing in vivo cancer or a MALT1-mediated disease in a subject, wherein the dose and/or frequency of dosing for the MALT1 inhibitor is modified by the method comprising:

(a) measuring a changed level of NF-kB nuclear translocation in a subject's test sample that has been previously exposed to the MALT1 inhibitor;

(b) measuring a changed level of NF-kB nuclear translocation in a subject's control sample that has not been previously exposed to the MALT1 inhibitor;

(c) comparing the changed level of NF-kB nuclear translocation in (a) to (b); and the method further comprises reducing a dosing frequency of the MALT1 inhibitor if the changed level of NF-kB nuclear translocation in (a) is less than (b), and increasing the dosing frequency of the MALT1 inhibitor if the changed level of NF-kB nuclear translocation in (a) is not less than (b).

6. The method of any one of embodiments 1-5 or the MALT1 inhibitor for use in any one of the embodiments 1a-5a, wherein measuring the changed level of NF-kB nuclear translocation in the subject's test sample comprises:

a) contacting a first portion of the test sample with one or more stimulating agents to obtain a stimulated test sample, and keeping a second portion of the test sample that is not contacted with the one or more stimulating agents as an unstimulated test sample;

b) measuring a first level of NF-kB nuclear translocation from cytoplasm into nucleus of the stimulated test sample;

c) measuring a second level of NF-kB nuclear translocation from cytoplasm into nucleus of the unstimulated test sample, wherein the cells from the stimulated sample and the unstimulated sample are of the same cell type; and

d) measuring the changed the level of NF-kB nuclear translocation in the test sample by comparing the first level of NF-kB nuclear translocation with the second level of NF-kB nuclear translocation.

7. The method of any one of embodiments 1-5 or the MALT1 inhibitor for use of any one of embodiments 1a-5a, wherein measuring the changed level of NF-kB nuclear translocation in the subject's control sample comprises:

a) contacting a first portion of the control sample with the one or more stimulating agents to obtain a stimulated control sample, and keeping a second portion of the control sample that is not contacted with the one or more stimulating agents as an unstimulated control sample;

b) measuring a third level of NF-kB nuclear translocation from cytoplasm into nucleus of the stimulated control sample;

c) measuring a fourth level of NF-kB nuclear translocation from cytoplasm into nucleus of the unstimulated control sample, wherein the cells from the stimulated sample and the unstimulated sample are of the same cell type; and

d) measuring the changed level of NF-kB nuclear translocation in the control sample by comparing the third level of NF-kB nuclear translocation with the fourth level of NF-kB nuclear translocation.

8. The method of any one of embodiments 3-5 or the MALT1 inhibitor for use of any one of embodiments 3a-5a, wherein the cancer is selected from non-Hodgkin's lymphoma, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), lymphoblastic T cell leukemia, chronic myelogenous leukemia (CML), small lymphocytic lymphoma (SLL), Waldenstrom macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocytic leukemia, promyelocytic leukemia, erytholeukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).
9. The method of any one of embodiments 3-5 or the MALT1 inhibitor for use of any one of embodiments 3a-5a, wherein the MALT1-mediated disease is an immunological disease selected from arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's disease, uveitis, myasthenia gravis, Grave's disease, Hashimoto thyroiditis, Sjoergen's syndrome, a blistering disorder, antibody-mediated vasculitis syndromes, immune-complex vasculitides, an allergic disorder, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.
10. The method or the MALT1 inhibitor for use of embodiments 6 or 7, wherein the one or more stimulating agents is selected from IL-1α, IL-1β, TNF-α, a lipopolysaccharide (LPS), exotoxin B, phorbol myristate acetate (PMA)/ionomycin, a TLR agonist, an anti-CD3 antibody, anti-CD8 antibody, anti-IgM antibody, and combinations thereof.
11. The method or the MALT1 inhibitor for use of embodiments 6 or 7, wherein the test sample or the control sample is contacted with one or more of the stimulating agents for about 1 to 12 hours, about 1 to 10 hours, about 1 to 9 hours, or about 1 to 8 hours.
12. The method or the MALT1 inhibitor for use of embodiments 6 or 7, wherein the NF-κB nuclear translocation from the cytoplasm into the nucleus of a cell in the subject's sample is measured by a fluorescence based assay selected from flow cytometry, preferably imaging flow cytometry (IFC), luminescent analysis, chemiluminescent analysis, histochemistry, and fluorescent microscopy.
13. The method of embodiment 4 or the MALT1 inhibitor for use of embodiment 4a, wherein the second therapeutic agent is selected from BTK (Bruton's tyrosine kinase) inhibitors, SYK inhibitors, PKC inhibitors, PI3K pathway inhibitors, BCL family inhibitors, JAK inhibitors, PIM kinase inhibitors, B cell antigen-binding antibodies, anti-PD1 antibodies, anti-PD-L1 antibodies, and combinations thereof.
14. The method or the MALT1 inhibitor for use of any one of embodiments 1-7 and embodiments 1a-5a, wherein the MALT1 inhibitor is a compound of Formula (I)

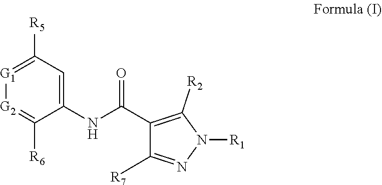

wherein
R₁ is selected from the group consisting of
i) naphthalen-1-yl, optionally substituted with a fluoro or amino substituent; and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, cyclopropyl, methoxymethyl, difluoromethyl, 1,1-difluoroethyl, hydroxymethyl, 1-hydroxyethyl, 1-ethoxyethyl, hydroxy, methoxy, ethoxy, fluoro, chloro, bromo, methylthio, cyano, amino, methylamino, dimethylamino, 4-oxotetrahydrofuran-2-yl, 5-oxopyrrolidin-2-yl, 1,4-dioxanyl, aminocarbonyl, methylcarbonyl, methylaminocarbonyl, oxo, 1-(t-butoxycarbonyl)azetidin-2-yl, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
R₂ is selected from the group consisting of C₁₋₄alkyl, 1-methoxy-ethyl, difluoromethyl, fluoro, chloro, bromo, cyano, and trifluoromethyl;
G₁ is N or C(R₄);
G₂ is N or C(R₃); such that only one of G₁ and G₂ are N in any instance;
R₃ is independently selected from the group consisting of trifluoromethyl, cyano, C₁₋₄alkyl, fluoro, chloro, bromo, methylcarbonyl, methylthio, methylsulfinyl, and methanesulfonyl; or, when G₁ is N, R₃ is further selected from C₁₋₄alkoxycarbonyl;
R₄ is selected from the group consisting of
i) hydrogen, when G₂ is N;
ii) C₁₋₄alkoxy;
iii) cyano;
iv) cyclopropyloxy;
v) a heteroaryl selected from the group consisting of triazolyl, oxazolyl, isoxazolyl, pyrazolyl, pyrrolyl, thiazolyl, tetrazolyl, oxadiazolyl, imidazolyl, 2-amino-pyrimidin-4-yl, 2H-[1,2,3]triazolo[4,5-c]pyridin-2-yl, 2H-[1,2,3]triazolo[4,5-b]pyridin-2-yl, 3H-[1,2,3]triazolo[4,5-b]pyridin-3-yl, 1H-[1,2,3]triazolo[4,5-c]pyridin-1-yl, wherein the heteroaryl is optionally substituted with one or two substituents independently selected from oxo, C₁₋₄alkyl, carboxy, methoxycarbonyl, aminocarbonyl, hydroxymethyl, aminomethyl, (dimethylamino)methyl, amino, methoxymethyl, trifluoromethyl, amino(C₂₋₄alkyl)amino, or cyano;
vi) 1-methyl-piperidin-4-yloxy;
vii) 4-methyl-piperazin-1-ylcarbonyl;
viii) (4-aminobutyl)aminocarbonyl;
ix) (4-amino)butoxy;
x) 4-(4-aminobutyl)-piperazin-1-ylcarbonyl;
xi) methoxycarbonyl;
xii) 5-chloro-6-(methoxycarbonyl)pyridin-3-ylaminocarbonyl;
xiii) 1,1-dioxo-isothiazolidin-2-yl;
xiv) 3-methyl-2-oxo-2,3-dihydro-1H-imidazol-1-yl;
xv) 2-oxopyrrolidin-1-yl;
xvi) (E)-(4-aminobut-1-en-1-yl-aminocarbonyl;
xvii) difluoromethoxy; and
xviii) morpholin-4-ylcarbonyl;
R₅ is independently selected from the group consisting of hydrogen, chloro, fluoro, bromo, methoxy, methylsulfonyl, cyano, C₁₋₄alkyl, ethynyl, morpholin-4-yl, trifluoromethyl, hydroxyethyl, methylcarbonyl, methylsulfinyl, 3-hydroxy-pyrrolidin-1-yl, pyrrolidin-2-yl, 3-hydroxyazetidinyl, azetidin-3-yl, azetidin-2-yl, methylthio, and 1,1-difluoroethyl;
or R₄ and R₅ can be taken together to form 8-chloro-4-methyl-3-oxo-3,4-dihydro-2H-benzo[b][1,4]oxazin-6-yl, 8-chloro-3-oxo-3,4-dihydro-2H-benzo[b][1,4]oxazin-6-yl, 2-methyl-1-oxo-1,2,3,4-tetrahydroisoquinolin-7-yl, 4-methyl-3-oxo-3,4-dihydro-2H-benzo[b][1,4]oxazin-6-yl, 3-oxo-3,4-dihydro-2H-benzo[b][1,4]oxazin-6-yl, 1-methyl-1H-pyrazolo[3,4-b]pyridin-5-yl, 1H-pyrazolo[3,4-b]pyridin-5-yl, 2,3-dihydro-[1,4]dioxino[2,3-b]pyridin-5-yl, 1,3-dioxolo[4,5]pyridine-5-yl, 1-oxo-1,3-dihydroisobenzofuran-5-yl, 2,2-dimethylbenzo[d][1,3]dioxol-5-yl, 2,3-dihydrobenzo[b][1,4]dioxin-6-yl, 1-oxoisoindolin-5-yl, or 2-methyl-1-oxoisoindolin-5-yl, 1H-indazol-5-yl;
R₆ is hydrogen, C₁₋₄alkyl, fluoro, 2-methoxy-ethoxy, chloro, cyano, or trifluoromethyl;
R₇ is hydrogen or fluoro;
provided that a compound of Formula (I) is other than
a compound wherein R₁ is isoquinolin-8-yl, R₂ is trifluoromethyl, G₁ is C(R₄) wherein R₄ is 2H-1,2,3-triazol-2-yl, G₂ is N, and R₅ is hydrogen;
a compound wherein R₁ is isoquinolin-8-yl, R₂ is trifluoromethyl, G₁ is C(R₄) wherein R₄ is 1H-imidazol-1-yl, G₂ is N, and R₅ is chloro;
a compound wherein R₁ is isoquinolin-8-yl, R₂ is trifluoromethyl, G₁ is C(R₄) wherein R₄ is 1H-1,2,3-triazol-1-yl, G₂ is N, and R₅ is hydrogen;
a compound wherein R₁ is isoquinolin-8-yl, R₂ is trifluoromethyl, G₁ is C(R₄) wherein R₄ is hydrogen, G₂ is N, and R₅ is fluoro; or
an enantiomer, diastereomer, solvate, or pharmaceutically acceptable salt form thereof.
   15. The method or the MALT1 inhibitor for use of embodiment 14, wherein the MALT1 inhibitor is 1-(1-oxo-1,2 dihydroisoquinolin-5-yl)-5 (trifluoromethyl)-N-[2 (trifluoromethyl)pyridin-4 yl]-1H-pyrazole-4 carboxamide, represented by Formula (II):

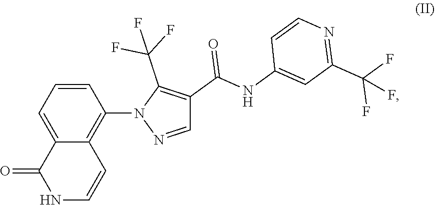 [SMILES: O=C(Nc1ccnc(C(F)(F)F)c1)c1cnn(-c2cccc3c(=O)[nH]ccc23)c1C(F)(F)F]

or a solvate, a tautomer, or a pharmaceutically acceptable salt thereof.
16. A method of treating cancer or a MALT1-mediated disease in a subject in need thereof, or a MALT1 inhibitor for use in a method of treating cancer or a MALT1-mediated disease in a subject, comprising:

a) contacting a first portion of a subject's test blood sample with one or more stimulating agents to obtain a stimulated sample, and keeping a second portion of a subject's test blood sample that is not contacted with the one or more stimulating agents as an unstimulated sample, and wherein the test blood sample has been previously exposed to a MALT1 inhibitor;

b) measuring a first level of NF-κB nuclear translocation from cytoplasm into nucleus of PBMCs in the stimulated sample;

c) measuring a second level of NF-κB nuclear translocation from cytoplasm into nucleus of PBMCs in the unstimulated sample, wherein the PBMCs in the unstimulated sample and the stimulated sample are of the same cell type;

d) comparing the first level with the second level to obtain a changed level of NF-κB nuclear translocation in the test blood sample;

e) comparing the changed level of NF-κB nuclear translocation in the test blood sample with a changed level of NF-κB nuclear translocation in a control blood sample, and

f) if the test sample does not display a decrease in the changed level of NF-kB nuclear translocation, then administering a dose of MALT1 inhibitor to the subject from about 1 mg to about 1000 mg.

17. A method of modifying the dose and/or frequency of dosing of a MALT1 inhibitor in a subject suffering from cancer or a MALT1-mediated disease, or a MALT1 inhibitor for use in a method of treating cancer or a MALT1-mediated disease in a subject, wherein the dose and/or frequency of dosing for the MALT1 inhibitor is modified by a method, comprising:

a) contacting a first portion of a subject's test blood sample with one or more stimulating agents to obtain a stimulated sample, and keeping a second portion of a subject's test blood sample that is not contacted with the one or more stimulating agents as an unstimulated sample, and wherein the test blood sample has been previously exposed to a MALT1 inhibitor;

b) measuring a first level of NF-κB nuclear translocation from cytoplasm into nucleus of PBMCs in the stimulated sample;

c) measuring a second level of NF-κB nuclear translocation from cytoplasm into nucleus of PBMCs in the unstimulated sample, wherein the PBMCs in the unstimulated sample and the stimulated sample are of the same cell type;

d) comparing the first level with the second level to obtain a changed level of NF-κB nuclear translocation in the test blood sample;

e) comparing the changed level of NF-κB nuclear translocation in the test blood sample with a changed level of NF-κB nuclear translocation in a control blood sample, and

f) if the test sample does not display a decrease in the changed level of NF-kB nuclear translocation, then administering an effective amount of MALT1 inhibitor to the subject from about 1 mg/day to about 1000 mg/day.

18. A method of assessing the pharmacodynamic effects of a MALT1 inhibitor in a human subject in need of a treatment of a MALT1-mediated disease, the method comprising detecting a suppression by the MALT1 inhibitor of NF-κB nuclear translocation in a stimulated peripheral blood mononuclear cell (PBMC) of a blood sample of the subject, wherein the blood sample has been treated with one or more stimulating agents in vitro prior to the detecting of the suppression.
19. A MALT1 inhibitor for use in a method of treating and/or diagnosing in vivo a MALT1-mediated disease in a human subject, wherein the MALT1 inhibitor is determined to be efficacious in the subject or the subject is determined to be responsive to the MALT1 inhibitor by the method comprising:

detecting a suppression by the MALT1 inhibitor of NF-κB nuclear translocation in a stimulated peripheral blood mononuclear cell (PBMC) of a blood sample of the subject, wherein the blood sample has been treated with one or more stimulating agents in vitro prior to the detecting of the suppression;

wherein the MALT1 inhibitor is determined to be efficacious in treating the MALT1-mediated disease in the subject or the subject is determined to be responsive to a treatment with the MALT1 inhibitor if the suppression is detected.

20. A method for assessing the pharmacodynamic effects of a MALT1 inhibitor in a subject in need of a treatment of a MALT1-mediated disease, comprising:

a) administering to a first portion of a blood sample of the subject one or more stimulating agents to thereby obtain a stimulated sample, and keeping a second portion of the blood sample not administered with the one or more stimulating agents as an unstimulated sample, wherein the MALT1 inhibitor has been administered to the subject or to the blood sample of the subject;

b) measuring a first level of NF-κB nuclear translocation from the cytoplasm into the nucleus of a stimulated PBMC in the stimulated sample;

c) measuring a second level of NF-κB nuclear translocation from the cytoplasm into the nucleus of an unstimulated PBMC in the unstimulated sample, wherein the unstimulated PBMC is of the same cell type of the stimulated PBMC;

d) comparing the first level with the second level to thereby determine a changed level of NF-κB nuclear translocation in the stimulated PBMC upon stimulation with the one or more stimulating agents in the presence of the MALT1 inhibitor; and

e) comparing the changed level of NF-κB nuclear translocation with a control to detect a suppression by the MALT1 inhibitor of the NF-κB nuclear translocation in the stimulated PBMC,

wherein the MALT1 inhibitor is determined to be efficacious in treating the MALT1-mediated disease in the subject or the subject is determined to be responsive to a treatment with the MALT1 inhibitor if the suppression is detected.

21. A MALT1 inhibitor for use in a method of treating and/or diagnosing in vivo a MALT1-mediated disease in a human subject, wherein the MALT1 inhibitor is determined to be efficacious in the subject or the subject is determined to be responsive to the MALT1 inhibitor by the method comprising:

a) administering to a first portion of a blood sample of the subject one or more stimulating agents to thereby obtain a stimulated sample, and keeping a second portion of the blood sample not administered with the one or more stimulating agents as an unstimulated sample, wherein the MALT1 inhibitor has been administered to the subject or to the blood sample of the subject;

b) measuring a first level of NF-κB nuclear translocation from the cytoplasm into the nucleus of a stimulated PBMC in the stimulated sample;

c) measuring a second level of NF-κB nuclear translocation from the cytoplasm into the nucleus of an unstimulated PBMC in the unstimulated sample, wherein the unstimulated PBMC is of the same cell type of the stimulated PBMC;

d) comparing the first level with the second level to thereby determine a changed level of NF-κB nuclear translocation in the stimulated PBMC upon stimulation with the one or more stimulating agents in the presence of the MALT1 inhibitor; and

e) comparing the changed level of NF-κB nuclear translocation with a control to detect a suppression by the MALT1 inhibitor of the NF-κB nuclear translocation in the stimulated PBMC,

wherein the MALT1 inhibitor is determined to be efficacious in treating the MALT1-mediated disease in the subject or the subject is determined to be responsive to a treatment with the MALT1 inhibitor if the suppression is detected.

22. The method of embodiment 20 or the MALT1 inhibitor for use of embodiment 21, wherein the control corresponds to a changed level of NF-κB nuclear translocation in a stimulated control PBMC upon stimulation with the one or more stimulating agents in the absence of the MALT1 inhibitor, preferably the control is measured by a method comprising:

a) administering to a first portion of a control blood sample of the subject the one or more stimulating agents to thereby obtain a stimulated control sample, and keeping a second portion of the control blood sample not administered with the one or more stimulating agents as an unstimulated control sample, wherein the MALT1 inhibitor has not been administered to the control blood sample either in vivo or in vitro;

b) measuring a third level of NF-κB nuclear translocation from the cytoplasm into the nucleus of the stimulated control PBMC in the stimulated control sample;

c) measuring a fourth level of the NF-κB nuclear translocation from the cytoplasm into the nucleus of an unstimulated control PBMC in the unstimulated control sample, wherein the stimulated control PBMC and the unstimulated control PBMC are of the same cell type of the stimulated PBMC; and

d) comparing the third level with the fourth level to thereby determine the changed level of the NF-κB nuclear translocation in the stimulated control PBMC upon stimulation with the one or more stimulating agents in the absence of the MALT1 inhibitor.

23. The method or the MALT1 inhibitor for use of any one of embodiments 18 to 22, wherein the stimulated PBMC is a T cell, B cell, natural killer cell, monocyte, or dendritic cell.
24. The method or the MALT1 inhibitor for use of any one of embodiments 18-23, wherein the MALT1-mediated disease is a lymphoma, such as a non-Hodgkin lymphoma (NHL), preferably a diffuse large B-cell lymphoma (DLBCL), more preferably an activated B-cell-like (ABC) subtype of DLBCL, or the MALT1-mediated disease is a leukemia, preferably a chronic lymphocytic leukemia (CLL).
25. A MALT1 inhibitor for use in a method of treating and/or diagnosing in vivo a MALT1-mediated disease, wherein the MALT1-mediated disease is a lymphoma, such as an NHL, or a leukemia, such as a CLL in a human subject, wherein the MALT1 inhibitor is determined to be efficacious in the subject or the subject is determined to be responsive to the MALT1 inhibitor by the method comprising:

a) administering to a first portion of a blood sample of the subject at least one of an anti-CD3 antibody and an anti-CD28 antibody or antigen binding fragments thereof, preferably both the anti-CD3 antibody and the anti-CD28 antibody or antigen binding fragments thereof, to thereby obtain a first stimulated sample, and keeping a second portion of the blood sample not administered with the at least one of the anti-CD3 antibody and the anti-CD28 antibody or antigen binding fragments thereof as a first unstimulated sample, wherein the MALT1 inhibitor has been administered to the subject or the blood sample of the subject;

b) measuring a first level of NF-κB nuclear translocation from the cytoplasm into the nucleus of a stimulated T cell in the first stimulated sample;

c) measuring a second level of the NF-κB nuclear translocation from the cytoplasm into the nucleus of an unstimulated T cell in the first unstimulated sample;

d) comparing the first level with the second level to thereby determine a changed level of NF-κB nuclear translocation in the stimulated T cell upon stimulation with the at least one of the anti-CD3 antibody and the anti-CD28 antibody or antigen binding fragments thereof in the presence of the MALT1 inhibitor; and

e) comparing the changed level of NF-κB nuclear translocation with a first control to detect a suppression by the MALT1 inhibitor of the NF-κB nuclear translocation in the stimulated T cell,

wherein the MALT1 inhibitor is determined to be efficacious in treating the MALT1-mediated disease in the subject or the subject is determined to be responsive to a treatment with the MALT1 inhibitor if the suppression is detected.

26. The MALT1 inhibitor for use of embodiment 25, wherein the first control corresponds to a changed level of NF-κB nuclear translocation in a stimulated control T cell upon stimulation with the at least one of an anti-CD3 antibody and an anti-CD28 antibody or antigen binding fragments thereof in the absence of the MALT1 inhibitor, preferably the first control is measured by a method comprising:

a) administering to a first portion of a first control blood sample of the subject the at least one of an anti-CD3 antibody and an anti-CD28 antibody or antigen binding fragments thereof to thereby obtain a first stimulated control sample, and keeping a second portion of the first control blood sample not administered with the one or more stimulating agents as a first unstimulated control sample, wherein the MALT1 inhibitor has not been administered to the first control blood sample either in vivo or in vitro;

b) measuring a third level of NF-κB nuclear translocation from the cytoplasm into the nucleus of the stimulated control T cell in the first stimulated control sample;

c) measuring a fourth level of the NF-κB nuclear translocation from the cytoplasm into the nucleus of an unstimulated control T cell in the first unstimulated control sample; and d) comparing the third level with the fourth level to thereby determine the changed level of the NF-κB nuclear translocation in the stimulated control T cell upon stimulation with the at least one of an anti-CD3 antibody and an anti-CD28 antibody or antigen binding fragments thereof in the absence of the MALT1 inhibitor.

27. The method or the MALT1 inhibitor for use of any one of embodiments 25-26, wherein the anti-CD3 antibody and the anti-CD28 antibody or antigen binding fragments thereof are administered to and incubated with the first portion of the blood sample or the first portion of the control blood sample for about 1 to 9 hours, such as about 1, 2, 3, 4, 5, 6, 7, 8 or 9 hours, preferably about 5 to 6 hours, at 37° C., to obtain the first stimulated sample or the first stimulated control sample, respectively.
28. The method or the MALT1 inhibitor for use of any one of embodiments 25-27, wherein the MALT1-mediated disease is the NHL, and the method further comprises:

a) measuring a first CD69 expression level from a stimulated T cell in the first stimulated sample;

b) measuring a second CD69 expression level from an unstimulated T cell in the first unstimulated sample;

c) comparing the first CD69 expression level with the second CD69 expression level to thereby determine a changed level of CD69 expression in the stimulated T cell upon stimulation with the at least one of the anti-CD3 antibody and the anti-CD28 antibody or antigen binding fragments thereof in the presence of the MALT1 inhibitor; and

d) comparing the changed level of CD69 expression level determined in (c) with a second control to further assess the pharmacodynamic effects of the MALT1 inhibitor in the subject,

wherein when the changed level of CD69 expression in the stimulated T cell upon stimulation with the at least one of the anti-CD3 antibody and the anti-CD28 antibody or antigen binding fragments thereof in the presence of the MALT1 inhibitor is less than the second control, the MALT1 inhibitor is further determined to be efficacious in treating the NHL in the subject or the subject is further determined to be responsive to a treatment with the MALT1 inhibitor, preferably, the CD69 expression level is determined by flow cytometry, more preferably, the CD69 expression level is determined by imaging flow cytometry.

29. A method or the MALT1 inhibitor for use of any one of the other embodiments, wherein the MALT1 inhibitor is determined to be efficacious in treating the MALT1-mediated disease in the subject or the subject is determined to be responsive to a treatment with the MALT1 inhibitor if the suppression is detected.
30. A kit or combination for assessing the pharmacodynamic effects of a MALT1 inhibitor in a human subject in need of a treatment of a MALT1-mediated disease, comprising:

(1) one or more agents for stimulating a PBMC in a blood sample;

(2) an agent for fixing the PBMC;

(3) a labeled antibody against a surface antigen specific to the PBMC;

(4) an agent for permeabilizing the PBMC;

(5) an agent for staining the nuclear of the PBMC; and

(6) a labeled antibody specific for NF-κB.

31. The kit of embodiment 30 for assessing the pharmacodynamic effects of the MALT1 inhibitor in the human subject in need of a treatment of an NHL, preferably diffuse large B-cell lymphoma (DLBCL), more preferably activated B-cell-like (ABC) subtype of DLBCL, or a leukemia, preferably CLL, comprising:

(1) at least one of an anti-CD3 antibody and an anti-CD28 antibody or antigen binding fragments thereof for stimulating T cells in the blood sample;

(2) a fluorescent labeled anti-CD4 antibody and a fluorescent labeled anti-CD8 antibody for detecting T cells activated by the at least one of anti-CD3 antibody and anti-CD8 antibody;

(3) the agent for fixing the T cells, preferably 4.21% formaldehyde (BD Pharmingen, cat. 554655);

(4) the agent for permeabilizing the T cells, preferably selected from the group consisting of Triton X-100, Tween 20, saponin, digitonin, and methanol;

(5) the agent for staining the nuclear of the T cells, preferably selected from the group consisting of DAPI, propidium iodide, DRAQ5, DRAQ7, and Hoescht stain; and (6) a fluorescent labeled antibody specific to p50, p65, RelB, c-Rel, p105/p50 or p100/52, preferably fluorescent labeled anti-p50 antibody.

32. The kit of embodiment 30 or 31 for assessing the pharmacodynamic effects of the MALT1 inhibitor in the human subject in need of a treatment of an NHL, further comprising a fluorescent labeled anti-CD69 antibody for measuring the CD69 expression level.
33. The kit of embodiment 30 or 31 for assessing the pharmacodynamic effects of the MALT1 inhibitor in the human subject in need of a treatment of a CLL, further comprising:

(1) an anti-IgM antibody or antigen binding fragment thereof for stimulating B cells in the blood sample; and

(2) a fluorescent labeled anti-CD19 antibody or anti-CD20 antibody for detecting activated B cells.

We claim:

1. A method of predicting a response to a MALT1 inhibitor in a subject in need thereof comprising:
(a) measuring a changed level of NF-kB nuclear translocation in a subject's test sample that has been previously exposed to the MALT1 inhibitor;
(b) measuring a changed level of NF-kB nuclear translocation in a subject's control sample that has not been previously exposed to the MALT1 inhibitor; and
(c) comparing the changed level of NF-kB nuclear translocation in (a) to (b), wherein a decrease in the changed level of NF-kB nuclear translocation in (a) is predictive of a positive response to the MALT1 inhibitor in the subject.

2. A method of monitoring an efficacy of an ongoing MALT1 inhibitor therapy in a subject in need thereof comprising:
(a) measuring a changed level of NF-kB nuclear translocation in a subject's test sample that has been previously exposed to a MALT1 inhibitor;
(b) measuring a changed level of NF-kB nuclear translocation in a subject's control sample that has not been previously exposed to a MALT1 inhibitor; and
(c) comparing the changed level of NF-kB nuclear translocation in (a) to (b), wherein a decrease in the changed level of NF-kB nuclear translocation in (a) is indicative of efficacy of the MALT1 inhibitor therapy in the subject.

3. A method of treating a cancer or a MALT1-mediated disease in a subject in need thereof comprising:
(a) measuring a changed level of NF-kB nuclear translocation in a subject's test sample that has been previously exposed to a MALT1 inhibitor;
(b) measuring a changed level of NF-kB nuclear translocation in a subject's control sample that has not been previously exposed to a MALT1 inhibitor;
(c) comparing the changed level of NF-kB nuclear translocation in (a) to (b); and
(d) administering a lower dose of MALT1 inhibitor to the subject if the test sample displays a decrease in the changed level of NF-kB nuclear translocation, and administering a higher dose of MALT1 inhibitor to the subject if the test sample does not display a decrease in the changed level of NF-kB nuclear translocation.

4. A method of designing a drug regimen to treat cancer or a MALT1-mediated disease in a subject in need thereof comprising:
(a) measuring a changed level of NF-kB nuclear translocation in a subject's test sample that has been previously exposed to a MALT1 inhibitor;
(b) measuring a changed level of NF-kB nuclear translocation in a subject's control sample that has not been previously exposed to a MALT1 inhibitor;
(c) comparing the changed level of NF-kB nuclear translocation in the subject's test sample to the changed level in the subject's control sample; and
(d) administering a second therapeutic agent to the subject if the test sample does not display a decrease in the changed level of NF-kB nuclear translocation.

5. A method of modifying the dose and/or frequency of dosing of a MALT1 inhibitor in a subject suffering from cancer or a MALT1-mediated disease comprising:
(a) measuring a changed level of NF-kB nuclear translocation in a subject's test sample that has been previously exposed to the MALT1 inhibitor;
(b) measuring a changed level of NF-kB nuclear translocation in a subject's control sample that has not been previously exposed to the MALT1 inhibitor;
(c) comparing the changed level of NF-kB nuclear translocation in the subject's test sample to the changed level of the control sample; and
(d) reducing a dosing frequency of the MALT1 inhibitor if the test sample displays a decrease in the changed level of NF-kB nuclear translocation, and increasing the dosing frequency of the MALT1 inhibitor if the test sample does not display a decrease in the changed level of NF-kB nuclear translocation.

6. The method of claim 3, wherein measuring the changed level of NF-kB nuclear translocation in the subject's test sample comprises:
a) contacting a first portion of the test sample with one or more stimulating agents to obtain a stimulated test sample, and keeping a second portion of the test sample that is not contacted with the one or more stimulating agents as an unstimulated test sample;
b) measuring a first level of NF-kB nuclear translocation from cytoplasm into nucleus of the stimulated test sample;
c) measuring a second level of NF-kB nuclear translocation from cytoplasm into nucleus of the unstimulated test sample, wherein the cells from the stimulated sample and the unstimulated sample are of the same cell type; and
d) measuring the changed the level of NF-kB nuclear translocation in the test sample by comparing the first level of NF-kB nuclear translocation with the second level of NF-kB nuclear translocation.

7. The method of claim 3, wherein measuring the changed level of NF-kB nuclear translocation in the subject's control sample comprises:
a) contacting a first portion of the control sample with the one or more stimulating agents to obtain a stimulated control sample, and keeping a second portion of the control sample that is not contacted with the one or more stimulating agents as an unstimulated control sample;
b) measuring a third level of NF-kB nuclear translocation from cytoplasm into nucleus of the stimulated control sample;
c) measuring a fourth level of NF-kB nuclear translocation from cytoplasm into nucleus of the unstimulated control sample, wherein the cells from the stimulated sample and the unstimulated sample are of the same cell type; and
d) measuring the changed level of NF-kB nuclear translocation in the control sample by comparing the third level of NF-kB nuclear translocation with the fourth level of NF-kB nuclear translocation.

8. The method of claim 3, wherein the cancer is selected from non-Hodgkin's lymphoma, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), lymphoblastic T cell leukemia, chronic myelogenous leukemia (CML), small lymphocytic lymphoma (SLL), Waldenstrom macroglobulinemia, lymphoblastic T cell leukemia, chronic myelogenous leukemia (CVL), hairy-cell leukemia, acute lymphoblastic T cell leukemia, plasmacytoma, immunoblastic large cell leukemia, megakaryoblastic leukemia, acute megakaryocytic leukemia, promyelocytic leukemia, erytholeukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

9. The method of claim 3, wherein the MALT1-mediated disease is an immunological disease selected from arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's disease, uveitis, myasthenia gravis, Grave's disease, Hashimoto thyroiditis, Sjoergen's syndrome, a blistering disorder, antibody-mediated vasculitis syndromes, immune-complex vasculitides, an allergic disorder, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

10. The method of claim 6, wherein the one or more stimulating agents is selected from IL-1α, IL-1β, TNF-α, a lipopolysaccharide (LPS), exotoxin B, phorbol myristate acetate (PMA)/ionomycin, a TLR agonist, an anti-CD3 antibody, anti-CD8 antibody, anti-IgM antibody, and combinations thereof.

11. The method of claim 6, wherein the test sample or the control sample is contacted with one or more of the stimulating agents for about 1 to 12 hours, about 1 to 10 hours, about 1 to 9 hours, or about 1 to 8 hours.

12. The method of claim 6, wherein the NF-κB nuclear translocation from the cytoplasm into the nucleus of a cell in the subject's sample is measured by a fluorescence based assay selected from flow cytometry, preferably imaging flow cytometry (IFC), luminescent analysis, chemiluminescent analysis, histochemistry, and fluorescent microscopy.

13. The method of claim 4, wherein the second therapeutic agent is selected from BTK (Bruton's tyrosine kinase) inhibitors, SYK inhibitors, PKC inhibitors, PI3K pathway inhibitors, BCL family inhibitors, JAK inhibitors, PIM kinase inhibitors, B cell antigen-binding antibodies, anti-PD1 antibodies, anti-PD-L1 antibodies, and combinations thereof.

14. The method of claim 3, wherein the MALT1 inhibitor is a compound of Formula (I) 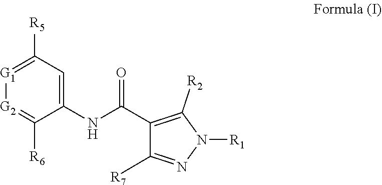
wherein
R₁ is selected from the group consisting of
i) naphthalen-1-yl, optionally substituted with a fluoro or amino substituent; and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, cyclopropyl, methoxymethyl, difluoromethyl, 1,1-difluoroethyl, hydroxymethyl, 1-hydroxyethyl, 1-ethoxyethyl, hydroxy, methoxy, ethoxy, fluoro, chloro, bromo, methylthio, cyano, amino, methylamino, dimethylamino, 4-oxotetrahydrofuran-2-yl, 5-oxopyrrolidin-2-yl, 1,4-dioxanyl, aminocarbonyl, methylcarbonyl, methylaminocarbonyl, oxo, 1-(t-butoxycarbonyl)azetidin-2-yl, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
R₂ is selected from the group consisting of C₁₋₄alkyl, 1-methoxy-ethyl, difluoromethyl, fluoro, chloro, bromo, cyano, and trifluoromethyl;
G₁ is N or C(R₄);
G₂ is N or C(R₃); such that only one of G₁ and G₂ are N in any instance;
R₃ is independently selected from the group consisting of trifluoromethyl, cyano, C₁₋₄alkyl, fluoro, chloro, bromo, methylcarbonyl, methylthio, methylsulfinyl, and methanesulfonyl;
or, when G₁ is N, R₃ is further selected from C₁₋₄alkoxycarbonyl;
R₄ is selected from the group consisting of
i) hydrogen, when G₂ is N;
ii) C₁₋₄alkoxy;
iii) cyano;
iv) cyclopropyloxy;
v) a heteroaryl selected from the group consisting of triazolyl, oxazolyl, isoxazolyl, pyrazolyl, pyrrolyl, thiazolyl, tetrazolyl, oxadiazolyl, imidazolyl, 2-amino-pyrimidin-4-yl, 2H-[1,2,3]triazolo[4,5-c]pyridin-2-yl, 2H-[1,2,3]triazolo[4,5-b]pyridin-2-yl, 3H-[1,2,3]triazolo[4,5-b]pyridin-3-yl, 1H-[1,2,3]triazolo[4,5-c]pyridin-1-yl, wherein the heteroaryl is optionally substituted with one or two substituents independently selected from oxo, C₁₋₄alkyl, carboxy, methoxycarbonyl, aminocarbonyl, hydroxymethyl, aminomethyl, (dimethylamino)methyl, amino, methoxymethyl, trifluoromethyl, amino(C₂₋₄alkyl)amino, or cyano;
vi) 1-methyl-piperidin-4-yloxy;
vii) 4-methyl-piperazin-1-ylcarbonyl;
viii) (4-aminobutyl)aminocarbonyl;
ix) (4-amino)butoxy;
x) 4-(4-aminobutyl)-piperazin-1-ylcarbonyl;
xi) methoxycarbonyl;
xii) 5-chloro-6-(methoxycarbonyl)pyridin-3-ylaminocarbonyl;
xiii) 1,1-dioxo-isothiazolidin-2-yl;
xiv) 3-methyl-2-oxo-2,3-dihydro-1H-imidazol-1-yl;
xv) 2-oxopyrrolidin-1-yl;
xvi) (E)-(4-aminobut-1-en-1-yl-aminocarbonyl;
xvii) difluoromethoxy; and
xviii) morpholin-4-ylcarbonyl;
R₅ is independently selected from the group consisting of hydrogen, chloro, fluoro, bromo, methoxy, methylsulfonyl, cyano, C₁₋₄alkyl, ethynyl, morpholin-4-yl, trifluoromethyl, hydroxyethyl, methylcarbonyl, methylsulfinyl, 3-hydroxy-pyrrolidin-1-yl, pyrrolidin-2-yl, 3-hydroxyazetidinyl, azetidin-3-yl, azetidin-2-yl, methylthio, and 1,1-difluoroethyl;
or R₄ and R₅ can be taken together to form 8-chloro-4-methyl-3-oxo-3,4-dihydro-2H-benzo[b][1,4]oxazin-6-yl, 8-chloro-3-oxo-3,4-dihydro-2H-benzo[b][1,4]oxazin-6-yl, 2-methyl-1-oxo-1,2,3,4-tetrahydroisoquinolin-7-yl, 4-methyl-3-oxo-3,4-dihydro-2H-benzo[b][1,4]oxazin-6-yl, 3-oxo-3,4-dihydro-2H-benzo[b][1,4]oxazin-6-yl, 1-methyl-1H-pyrazolo[3,4-b]pyridin-5-yl, 1H-pyrazolo[3,4-b]pyridin-5-yl, 2,3-dihydro-[1,4]dioxino[2,3-b]pyridin-5-yl, 1,3-dioxolo[4,5]pyridine-5-yl, 1-oxo-1,3-dihydroisobenzofuran-5-yl, 2,2-dimethylbenzo[d][1,3]dioxol-5-yl, 2,3-dihydrobenzo[b][1,4]dioxin-6-yl, 1-oxoisoindolin-5-yl, or 2-methyl-1-oxoisoindolin-5-yl, 1H-indazol-5-yl;
R₆ is hydrogen, C₁₋₄alkyl, fluoro, 2-methoxy-ethoxy, chloro, cyano, or trifluoromethyl;
R₇ is hydrogen or fluoro;
provided that a compound of Formula (I) is other than
a compound wherein R₁ is isoquinolin-8-yl, R₂ is trifluoromethyl, G₁ is C(R₄) wherein R₄ is 2H-1,2,3-triazol-2-yl, G₂ is N, and R₅ is hydrogen;
a compound wherein R₁ is isoquinolin-8-yl, R₂ is trifluoromethyl, G₁ is C(R₄) wherein R₄ is 1H-imidazol-1-yl, G₂ is N, and R₅ is chloro;
a compound wherein R₁ is isoquinolin-8-yl, R₂ is trifluoromethyl, G₁ is C(R₄) wherein R₄ is 1H-1,2,3-triazol-1-yl, G₂ is N, and R₅ is hydrogen;
a compound wherein R₁ is isoquinolin-8-yl, R₂ is trifluoromethyl, G₁ is C(R₄) wherein R₄ is hydrogen, G₂ is N, and R₅ is fluoro;
or an enantiomer, diastereomer, solvate, or pharmaceutically acceptable salt form thereof.

15. The method of claim 14, wherein the MALT1 inhibitor is 1-(1 oxo-1,2 dihydroisoquinolin-5 yl)-5 (trifluoromethyl)-N-[2 (trifluoromethyl)pyridin-4 yl]-1H-pyrazole-4 carboxamide, represented by Formula (II): 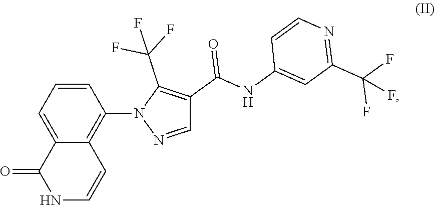 [SMILES: O=C(Nc1ccnc(C(F)(F)F)c1)c1cnn(-c2cccc3c(=O)[nH]ccc23)c1C(F)(F)F]
or a solvate, a tautomer, or a pharmaceutically acceptable salt thereof.

16. A method of treating cancer or a MALT1-mediated disease in a subject comprising:
a) contacting a first portion of a subject's test blood sample with one or more stimulating agents to obtain a stimulated sample, and keeping a second portion of a subject's test blood sample that is not contacted with the one or more stimulating agents as an unstimulated sample, and wherein the test blood sample has been previously exposed to a MALT1 inhibitor;
b) measuring a first level of NF-κB nuclear translocation from cytoplasm into nucleus of PBMCs in the stimulated sample;
c) measuring a second level of NF-κB nuclear translocation from cytoplasm into nucleus of PBMCs in the unstimulated sample, wherein the PBMCs in the unstimulated sample and the stimulated sample are of the same cell type;
d) comparing the first level with the second level to obtain a changed level of NF-κB nuclear translocation in the test blood sample;
e) comparing the changed level of NF-κB nuclear translocation in the test blood sample with a changed level of NF-κB nuclear translocation in a control blood sample, and
f) if the test sample does not display a decrease in the changed level of NF-kB nuclear translocation, then administering a dose of MALT1 inhibitor to the subject from about 1 mg to about 1000 mg.

17. A method of modifying the dose and/or frequency of dosing of a MALT1 inhibitor in a subject suffering from cancer or a MALT1-mediated disease comprising:
a) contacting a first portion of a subject's test blood sample with one or more stimulating agents to obtain a stimulated sample, and keeping a second portion of a subject's test blood sample that is not contacted with the one or more stimulating agents as an unstimulated sample, and wherein the test blood sample has been previously exposed to a MALT1 inhibitor;
b) measuring a first level of NF-κB nuclear translocation from cytoplasm into nucleus of PBMCs in the stimulated sample;
c) measuring a second level of NF-κB nuclear translocation from cytoplasm into nucleus of PBMCs in the unstimulated sample, wherein the PBMCs in the unstimulated sample and the stimulated sample are of the same cell type;
d) comparing the first level with the second level to obtain a changed level of NF-κB nuclear translocation in the test blood sample;
e) comparing the changed level of NF-κB nuclear translocation in the test blood sample with a changed level of NF-κB nuclear translocation in a control blood sample, and
f) if the test sample does not display a decrease in the changed level of NF-kB nuclear translocation, then administering an effective amount of MALT1 inhibitor to the subject from about 1 mg/day to about 1000 mg/day.
